(19) United States
(12) Patent Application Publication
(10) Pub. No.: US 2026/0160763 A1
(43) Pub. Date: Jun. 11, 2026

(54) METHODS AND MATERIALS FOR ASSESSING AND TREATING NEUROLOGIC AUTOIMMUNE DISORDERS AND/OR CANCER

(71) Applicant: Mayo Foundation for Medical Education and Research, Rochester, MN (US)

(72) Inventors: Anastasia Zekeridou, Rochester, MN (US); Vanda A. Lennon, Rochester, MN (US); Sean J. Pittock, Rochester, MN (US); Binxia Yang, Rochester, MN (US); Eoin P. Flanagan, Rochester, MN (US)

(21) Appl. No.: 18/708,005

(22) PCT Filed: Nov. 15, 2022
(86) PCT No.: PCT/US2022/079874

§ 371 Date: May 7, 2024

Related U.S. Application Data
(60) Provisional application No. 63/289,384, filed on Dec. 14, 2021.

(30) Foreign Application Priority Data
Nov. 16, 2021 (GR) 20210100802

(51) Int. Cl. G01N 33/564; G01N 33/544

(52) U.S. Cl. CPC G01N 33/564; G01N 33/544; G01N 2800/28

(57) ABSTRACT

This document relates to methods and materials involved in assessing and/or treating mammals having a neurological autoimmune disorder (e.g., a paraneoplastic neurological autoimmune disorder) and/or cancer. For example, methods and materials for detecting anti-neuronal nuclear antibody type 3 (ANNA3) antibodies are provided.

### CROSS-REFERENCE TO RELATED APPLICATIONS

This application claims the benefit of GR Patent Application Serial No. 20210100802, filed on Nov. 16, 2021, and of U.S. Patent Application Ser. No. 63/289,384, filed on Dec. 14, 2021. The disclosure of the prior applications are considered part of (and are incorporated by reference in) the disclosure of this application.

### SEQUENCE LISTING

This application contains a Sequence Listing that has been submitted electronically as an XML file named “07039-2087WO1.XML.” The XML file, created on Nov. 10, 2022, is 10000 bytes in size. The material in the XML file is hereby incorporated by reference in its entirety.

### TECHNICAL FIELD

This document relates to methods and materials involved in assessing and/or treating mammals having a neurologic autoimmune disorder (e.g., a paraneoplastic neurologic autoimmune disorder) and/or cancer. For example, this document relates to methods and materials for using a Dachshund homolog 1 (DACH1) polypeptide and/or one or more fragments of a DACH1 polypeptide to detect the presence or absence of anti-neuronal nuclear antibody type 3 (ANNA3) antibodies.

### BACKGROUND INFORMATION

Autoantibodies are increasingly validated as biomarkers for autoimmune diseases and cancers. For example, anti-neuronal nuclear antibody type 1 (ANNA1) antibodies and ANNA2 antibodies, specific for HuD and Nova1/Nova2 polypeptides, respectively, are recognized biomarkers of paraneoplastic autoimmune neurological disorders, and are incorporated into most serological evaluations of patients with suspected neurological autoimmunity (Lucchinetti et al., Neurology, 50:652-657 (1998); Pittock et al., Ann. Neurol., 53:580-587 (2003); and Pittock et al., Arch. Neurol., 67:1109-1115 (2010)). These IgG autoantibodies can also serve as biomarkers of cancer in patients who lack a neurological syndrome (Gozzard et al., PLoS One, 10:e0143558 (2015); Dalmau et al., Ann. Neurol., 27:544-552 (1990); Dalmau et al., Medicine (Baltimore), 71:59-72 (1992); Lucchinetti et al., Neurology, 50: 652-657 (1998); Graus et al., Neurosci. Lett., 150:212-214 (1993); Pittock et al., Ann. Neurol., 53:580-587 (2003); and Pittock et al., Arch. Neurol., 67:1109-1115 (2010)).

A third type of ANNA (ANNA3) antibody has also been described as a biomarker of neurological paraneoplastic autoimmunity. However, the ANNA3 antibody antigen was unknown. As such, detection of ANNA3 antibodies was based on the characteristic immunofluorescence staining on mouse tissue sections of the nuclei of the Purkinje neurons (most prominent), molecular and Golgi neurons (faint) and the glomerular podocytes in the renal cortex (prominent) (Chan et al. Ann. Neurol., 50:301-311 (2001)).

### SUMMARY

This document is based, at least in part, on the discovery that a DACH1 polypeptide is the antigen target of ANNA3 autoantibodies.

This document provides methods and materials for assessing and/or treating mammals (e.g., humans) having a neurologic autoimmune disorder (e.g., a paraneoplastic neurologic autoimmune disorder) and/or cancer. For example, this document provides methods and materials for detecting neurologic autoimmune disorders (e.g., paraneoplastic neurologic autoimmune disorders) and/or cancers associated with ANNA3 autoantibodies. As described herein, ANNA3 autoantibodies in mammals (e.g., humans) having a neurologic autoimmune disorder (e.g., a paraneoplastic neurologic autoimmune disorder) and/or cancer can target and bind to a DACH1 polypeptide. Accordingly, a DACH1 polypeptide and/or one or more fragments of a DACH1 polypeptide can be used to determine if a sample contains ANNA3 autoantibodies. For example, a sample (e.g., a serum sample) obtained from a mammal (e.g., a human) having a neurologic autoimmune disorder (e.g., a paraneoplastic neurologic autoimmune disorder) and/or cancer can be contacted with one or more fragments of a DACH1 polypeptide such that an ANNA3 antibody, if present, forms a complex with the fragment of the DACH1 polypeptide (an ANNA3 autoantibody-fragment complex). In some cases, a sample (e.g., a serum sample) obtained from a mammal (e.g., a human) having a neurologic autoimmune disorder (e.g., a paraneoplastic neurologic autoimmune disorder) and/or cancer can be contacted with cells designed to express a DACH1 polypeptide and/or lysate from cells designed to express a DACH1 polypeptide such that an ANNA3 antibody, if present, forms a complex with the DACH1 polypeptide (an ANNA3 autoantibody-DACH1 polypeptide complex). The presence of ANNA3 antibodies can be used to identify a mammal (e.g., a human) as having an ANNA3-positive neurologic autoimmune disorder and/or an ANNA3-positive cancer (e.g., as having cancer and as having the presence of ANNA3 autoantibodies in the blood or cerebrospinal fluid (CSF)).

Serological detection of ANNA3 autoantibodies can enable earlier diagnosis of an ANNA3-positive neurologic autoimmune disorder and/or an ANNA3-positive cancer, and can allow clinicians to provide appropriate treatment of paraneoplastic autoimmunity increasing the likelihood of optimal neurological outcomes and early cancer diagnosis leading to better oncological outcomes.

In general, one aspect of this document features methods for detecting an ANNA3 autoantibody in a sample. The methods can include, or consist essentially of, (a) contacting a sample with a composition comprising a DACH1 polypeptide, where at least 5 percent of the polypeptide content of the composition is the DACH1 polypeptide, to form an ANNA3 autoantibody-DACH1 polypeptide complex if the sample comprises the ANNA3 autoantibody; and (b) detecting the presence or absence of the complex, where the presence of the complex indicates that the sample contains the ANNA3 autoantibody, and where the absence of the complex indicates that the sample lacks the ANNA3 autoantibody. The composition can include a cell lysate obtained from a cell comprising exogenous nucleic acid encoding the DACH1 polypeptide and expressing the DACH1 polypeptide. The complex can be detecting using an immunological assay. The sample can be a blood sample (e.g., a serum sample) or a CSF sample. The sample can be obtained from a human.

In another aspect, this document features methods for detecting an ANNA3 autoantibody in a sample. The methods can include, or consist essentially of, (a) contacting a sample with a fragment of a DACH1 polypeptide to form an ANNA3 autoantibody-fragment complex if the sample comprises the ANNA3 autoantibody; and (b) detecting the presence or absence of the complex, where the presence of the complex indicates that the sample contains the ANNA3 autoantibody, and where the absence of the complex indicates that the sample lacks the ANNA3 autoantibody. The DACH1 polypeptide fragment can consist or consist essentially of the amino acid sequence set forth in any one of SEQ ID NOs:2-7. The DACH1 polypeptide fragment can be covalently linked to a detectable label. The detectable label can be tetramethylrhodamine isothiocyanate (TRITC), fluorescein isothiocyanate (FITC), a poly(His) tag, or a glutathione-S-transferase (GST) tag. The complex can be detecting using an immunological assay. The sample can be a blood sample (e.g., a serum sample) or a CSF sample. The sample can be obtained from a human.

In another aspect, this document features methods for assessing a mammal having an autoimmune neurological disorder. The methods can include, or consist essentially of, (a) determining the presence or absence of an ANNA3 autoantibody in a sample obtained from a mammal having an autoimmune neurological disorder, where the determining comprises: (i) contacting the sample with a composition including a DACH1 polypeptide, where at least 5 percent of the polypeptide content of the composition is the DACH1 polypeptide, to form an ANNA3 autoantibody-DACH1 polypeptide complex if the sample comprises the ANNA3 autoantibody, and (ii) detecting the presence or absence of the complex; (b) classifying the mammal as having an ANNA3-positive autoimmune neurological disorder if the complex is present; and (c) classifying the mammal as having an ANNA3-negative autoimmune neurological disorder if the complex is absent. The composition can include a cell lysate obtained from a cell comprising exogenous nucleic acid encoding the DACH1 polypeptide and expressing the DACH1 polypeptide. The mammal can be a human. The sample can be a blood sample (e.g., a serum sample) or a CSF sample. The autoimmune neurological disorder can be a paraneoplastic autoimmune neurological disorder. The method can include detecting the presence of the autoantibodies and classifying the mammal as having the ANNA3-positive autoimmune neurological disorder. The method can include detecting the absence of the autoantibodies and classifying the mammal as having the ANNA3-negative autoimmune neurological disorder.

In another aspect, this document features methods for assessing a mammal having an autoimmune neurological disorder. The methods can include, or consist essentially of, (a) determining the presence or absence of an ANNA3 autoantibody in a sample obtained from a mammal having an autoimmune neurological disorder, where the determining comprises: (i) contacting the sample with a fragment of a DACH1 polypeptide to form an ANNA3 autoantibody-fragment complex if the sample comprises the ANNA3 autoantibody, and (ii) detecting the presence or absence of the complex; (b) classifying the mammal as having an ANNA3-positive autoimmune neurological disorder if the complex is present; and (c) classifying the mammal as having an ANNA3-negative autoimmune neurological disorder if the complex is absent. The DACH1 polypeptide fragment can consist or consist essentially of the amino acid sequence set forth in any one of SEQ ID NOs:2-7. The mammal can be a human. The sample can be a blood sample (e.g., a serum sample) or a CSF sample. The autoimmune neurological disorder can be a paraneoplastic autoimmune neurological disorder. The method can include detecting the presence of the autoantibodies and classifying the mammal as having the ANNA3-positive autoimmune neurological disorder. The method can include detecting the absence of the autoantibodies and classifying the mammal as having the ANNA3-negative autoimmune neurological disorder.

In another aspect, this document features methods for treating a mammal having an autoimmune neurological disorder. The methods can include, or consist essentially of, (a) determining the presence of an ANNA3 autoantibody in a sample obtained from a mammal having an autoimmune neurological disorder, where the determining comprises: (i) contacting the sample with a composition comprising a DACH1 polypeptide, where at least 5 percent of the polypeptide content of the composition is the DACH1 polypeptide to form an ANNA3 autoantibody-DACH1 polypeptide complex if the sample comprises the ANNA3 autoantibody, and (ii) detecting the presence or absence of the complex, and (b) administering an immunosuppressant to the mammal. The composition can include a cell lysate obtained from a cell comprising exogenous nucleic acid encoding the DACH1 polypeptide and expressing the DACH1 polypeptide. The mammal can be a human. The sample can be a blood sample (e.g., a serum sample) or a CSF sample. The autoimmune neurological disorder can be a paraneoplastic autoimmune neurological disorder. The immunosuppressant can be cyclophosphamide, rituximab, azathioprine, mycophenolate mofetil, methotrexate, or a corticosteroid.

In another aspect, this document features methods for treating a mammal having an autoimmune neurological disorder. The methods can include, or consist essentially of, (a) determining the presence of an ANNA3 autoantibody in a sample obtained from a mammal having an autoimmune neurological disorder, where the determining comprises: (i) contacting the sample with a fragment of a DACH1 polypeptide to form an ANNA3 autoantibody-fragment complex if the sample comprises the ANNA3 autoantibody, and (ii) detecting the presence or absence of the complex; and (b) administering an immunosuppressant to the mammal. The DACH1 polypeptide fragment can consist or consist essentially of the amino acid sequence set forth in any one of SEQ ID NOs:2-7. The mammal can be a human. The sample can be a blood sample (e.g., a serum sample) or a CSF sample. The autoimmune neurological disorder can be a paraneoplastic autoimmune neurological disorder. The immunosuppressant can be cyclophosphamide, rituximab, azathioprine, mycophenolate mofetil, methotrexate, or a corticosteroid.

In another aspect, this document features methods for assessing a mammal having cancer. The methods can include, or consist essentially of, (a) determining the presence or absence of an ANNA3 autoantibody in a sample obtained from a mammal having cancer, where the determining comprises: (i) contacting the sample with a composition comprising a DACH1 polypeptide, where at least 5 percent of the polypeptide content of the composition is the DACH1 polypeptide to form an ANNA3 autoantibody-DACH1 polypeptide complex if the sample comprises the ANNA3 autoantibody, and (ii) detecting the presence or absence of the complex; (b) classifying the mammal as having an ANNA3-positive cancer if the complex is present; and (c) classifying the mammal as having an ANNA3-negative cancer if the complex is absent. The composition can include a cell lysate obtained from a cell comprising exogenous nucleic acid encoding the DACH1 polypeptide and expressing the DACH1 polypeptide. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The method can include detecting the presence of the autoantibodies and classifying the mammal as having the ANNA3-positive cancer. The method can include detecting the absence of the autoantibodies and classifying the mammal as having the ANNA3-negative cancer.

In another aspect, this document features methods for assessing a mammal having cancer. The methods can include, or consist essentially of, (a) determining the presence or absence of an ANNA3 autoantibody in a sample obtained from a mammal having cancer, where the determining comprises: (i) contacting the sample with a fragment of a DACH1 polypeptide to form an ANNA3 autoantibody-fragment complex if the sample comprises said ANNA3 autoantibody, and (ii) detecting the presence or absence of the complex; (b) classifying the mammal as having an ANNA3-positive cancer if the complex is present; and (c) classifying the mammal as having an ANNA3-negative cancer if the complex is absent. The DACH1 polypeptide fragment can consist or consist essentially of the amino acid sequence set forth in any one of SEQ ID NOs:2-7. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The method can include detecting the presence of the autoantibodies and classifying the mammal as having the ANNA3-positive cancer. The method can include detecting the absence of the autoantibodies and classifying the mammal as having the ANNA3-negative cancer.

In another aspect, this document features methods for treating a mammal having cancer. The methods can include, or consist essentially of, (a) determining the presence of an ANNA3 autoantibody in a sample obtained from a mammal having cancer, where the determining comprises: (i) contacting the sample with a composition including a DACH1 polypeptide, where at least 5 percent of the polypeptide content of the composition is the DACH1 polypeptide to form an ANNA3 autoantibody-DACH1 polypeptide complex if the sample comprises the ANNA3 autoantibody, and (ii) detecting the presence or absence of the complex, and (b) administering a cancer treatment to the mammal that is not an immune checkpoint inhibitor to the mammal. The composition can include a cell lysate obtained from a cell comprising exogenous nucleic acid encoding said DACH1 polypeptide and expressing said DACH1 polypeptide. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The immune checkpoint inhibitor can include ipilimumab, nivolumab, pembrolizumab, atezolizumab, avelumab, durvalumab, and/or cemiplimab.

In another aspect, this document features methods for treating a mammal having cancer. The methods can include, or consist essentially of, (a) determining the presence of an ANNA3 autoantibody in a sample obtained from a mammal having cancer, where the determining comprises: (i) contacting the sample with a fragment of a DACH1 polypeptide to form an ANNA3 autoantibody-fragment complex if the sample comprises the ANNA3 autoantibody, and (ii) detecting the presence or absence of the complex; and (b) administering a cancer treatment to the mammal that is not an immune checkpoint inhibitor to the mammal. The DACH1 polypeptide fragment can consist or consist essentially of the amino acid sequence set forth in any one of SEQ ID NOs:2-7. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The immune checkpoint inhibitor can include ipilimumab, nivolumab, pembrolizumab, atezolizumab, avelumab, durvalumab, and/or cemiplimab.

In another aspect, this document features methods for treating a mammal having cancer. The methods can include, or consist essentially of, (a) determining the absence of an ANNA3 autoantibody in a sample obtained from a mammal having cancer, where the determining comprises: (i) contacting the sample with a composition comprising a DACH1 polypeptide, where at least 5 percent of the polypeptide content of the composition is the DACH1 polypeptide to form an ANNA3 autoantibody-DACH1 polypeptide complex if the sample comprises the ANNA3 autoantibody, and (ii) detecting the absence of the complex, and (b) administering an immune checkpoint inhibitor to the mammal. The composition can include a cell lysate obtained from a cell comprising exogenous nucleic acid encoding the DACH1 polypeptide and expressing the DACH1 polypeptide. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The immune checkpoint inhibitor can include ipilimumab, nivolumab, pembrolizumab, atezolizumab, avelumab, durvalumab, and/or cemiplimab.

In another aspect, this document features methods for treating a mammal having cancer. The methods can include, or consist essentially of, (a) determining the absence of an ANNA3 autoantibody in a sample obtained from a mammal having cancer, where the determining comprises: (i) contacting the sample with a fragment of a DACH1 polypeptide to form an ANNA3 autoantibody-fragment complex if the sample comprises the ANNA3 autoantibody, and (ii) detecting the absence of the complex; and (b) administering an immune checkpoint inhibitor to the mammal. The DACH1 polypeptide fragment can consist or consist essentially of the amino acid sequence set forth in any one of SEQ ID NOs:2-7. The mammal can be a human. The sample can be a blood sample (e.g., a serum sample) or a CSF sample. The cancer can be a lung cancer, a bladder cancer, a teratoma, an ovarian cancer, a Merkel cell cancer, a gastric cancer, a colon cancer, or a breast cancer. The immune checkpoint inhibitor can include ipilimumab, nivolumab, pembrolizumab, atezolizumab, avelumab, durvalumab, and/or cemiplimab.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the methods, materials, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D. Discovery of ANNA3 antibodies in serum samples. FIGS. 1A and 1B) ANNA3-IgG binds selectively to nuclei in cerebellar Purkinje neurons and glomerular podocytes by indirect immunofluorescence on murine tissues (FIG. 1A), and to nuclei of the SCC37 small-cell carcinoma cell line (FIG. 1B). FIG. 1C) Western blot demonstrated ˜90 kDa immunoreactive band in SCC37 nuclear extract with 19/19 tested ANNA3-positive sera; control sera were non-reactive. *indicates patients with multiple specimens. FIG. 1D) IgG eluted from excised nitrocellulose region corresponding to the ˜90 kDa immunoreactive band yielded the ANNA3 immunostaining pattern when applied to mouse tissue sections; IgG eluted from the nitrocellulose region corresponding to ˜170 kDa did not. GL=granular layer; ML=molecular layer; Pc=Purkinje cells. Po=glomerular podocytes.

FIGS. 2A-2E. Confirmation of DACH1 as the antigenic target of ANNA3. FIG. 2A) By indirect immunofluorescence on murine tissues, IgG in ANNA3-positive patient serum co-localizes with rabbit anti-DACH1-IgG; no colocalization with control serum. FIG. 2B) Western blot shows binding of IgG in 25 ANNA3-positive sera to recombinant DACH1 (˜120 KDa) in lysate of transected HEK293 cells, which is identified by positive control rabbit anti-DACH1 IgG; all control patient IgGs are negative. FIG. 2C) Western shows binding of IgG in 25 ANNA3-positive sera to a recombinant DACH1 polypeptide (residues 282-758, ˜55 KDa, identified by positive control rabbit anti-DACH1 IgG); all control patient IgGs are negative. FIG. 2D) IgG in ANNA3-positive patient serum, but not in control serum, binds to permeabilized DACH1-GFP-transfected HEK293 cells. FIG. 2E) The immunostaining pattern typical of ANNA3-IgG was eliminated by pre-absorbing a positive patient's serum with lysate of DACH1 overexpressing HEK293 cells; the staining pattern of a control ANNA1-IgG positive patient's serum was not diminished by lysate absorption.

FIG. 3. DACH1-immunoreactivity in metastatic tumor cells of two ANNA3-positive patients. Case 1 (panels A-H): Resected mediastinal lymph node containing SCLC cells; H&E stain (panel A) and (panel E). The tumorous tissue is hypercellular with irregularly shaped tumor cells of increased nuclear: cytoplasmic ratio. The nuclei vary in size and exhibit coarse chromatin (panel E, inset). Synaptophysin (panel B and panel F) and chromogranin A (panel C and panel G) contour the tumor cells with a trabecular growth pattern typical of neuroendocrine differentiation. The nuclei of the tumor parenchymal cells are DACH1 immunoreactive (panel D and panel H). Case 2 (panels I-P): Resected right lower paratracheal lymph node tissue shows diffuse metastatic neoplastic cells infiltration (panel I and panel M). Tumor cells show obvious atypia with increased nuclear: cytoplasmic ratio, increased mitosis, and apoptosis. Synaptophysin (panel J and panel N) and TTF1 (panel K and panel O) immunoreactivities are consistent with neuroendocrine differentiation. The nuclei of the tumor parenchymal cells are immunoreactive for DACH1 (panel L and panel P). Normal lymph-node tissue (panels Q-S) shows no DACH1 immunoreactivity. Scale bars: 50 μm (panels A-D and R); 20 μm (panels E-H, M-P, and S); 100 μm (panel I-L and Q).

FIG. 4. An amino acid sequence of an exemplary DACH1 polypeptide (SEQ ID NO:1).

### DETAILED DESCRIPTION

A specific IgG autoantibody marker (ANNA3) is found in serum of some individuals having a neurologic autoimmune disorder (e.g., a paraneoplastic neurologic autoimmune disorder) and/or cancer. This document is based, at least in part, on the discovery that a DACH1 polypeptide is the antigen target of ANNA3 autoantibodies.

This document provides methods and materials for assessing and/or treating mammals (e.g., humans) having a neurologic autoimmune disorder (e.g., a paraneoplastic neurologic autoimmune disorder) and/or cancer. In some cases, this document provides materials and methods for using a DACH1 polypeptide and/or one or more fragments of a DACH1 polypeptide to detect ANNA3 autoantibodies in a mammal (e.g., a human having a neurologic autoimmune disorder and/or cancer). For example, this document provides methods and materials for detecting ANNA3 antibodies (e.g., ANNA3 autoantibodies) in a sample obtained from a mammal (e.g., a human) having a neurologic autoimmune disorder (e.g., a paraneoplastic neurologic autoimmune disorder) and/or cancer. In some cases, a sample (e.g., a serum sample) obtained from a mammal (e.g., a human) having a neurologic autoimmune disorder (e.g., a paraneoplastic neurologic autoimmune disorder) and/or cancer can be assessed for the presence or absence of ANNA3 antibodies (e.g., ANNA3 autoantibodies). The presence of ANNA3 autoantibodies can be used to identify the individual as having an ANNA3-positive neurologic autoimmune disorder and/or as having an ANNA3-positive cancer (e.g., as having cancer and as having the presence of ANNA3 autoantibodies). Also provided herein are materials and methods for treating mammals (e.g., humans) having a neurologic autoimmune disorder (e.g., a paraneoplastic neurologic autoimmune disorder) and/or cancer based, at least in part, on the presence or absence of ANNA3 antibodies (e.g., autoantibodies) in a sample (e.g., a serum sample) obtained from the mammal.

Any appropriate mammal having a neurologic autoimmune disorder (e.g., a paraneoplastic neurologic autoimmune disorder) and/or cancer can be assessed and/or treated as described herein. Examples of mammals that can have a neurologic autoimmune disorder and/or cancer and can be assessed for the presence or absence of ANNA3 antibodies (e.g., ANNA3 autoantibodies) as described herein include, without limitation, primates (e.g., humans and monkeys), dogs, cats, horses, cows, pigs, sheep, rabbits, mice, and rats. For example, humans having a neurologic autoimmune disorder (e.g., a paraneoplastic neurologic autoimmune disorder) can be assessed for the presence or absence of ANNA3 antibodies (e.g., ANNA3 autoantibodies) and, optionally, can be treated as described herein.

A mammal (e.g., human) having any type of neurologic autoimmune disorder can be assessed and/or treated as described herein. In some cases, a neurologic autoimmune disorder can be a paraneoplastic neurologic autoimmune disorder. Examples of neurologic autoimmune disorders that can be ANNA3-positive (e.g., can include the presence of ANNA3 autoantibodies) include, without limitation, autoimmune encephalitis, optic neuritis, myelitis, autoimmune cerebellar ataxia, immune-mediated neuropathies, and dysautonomia.

A mammal (e.g., human) having any type of cancer can be assessed and/or treated as described herein. In some cases, a cancer can be a primary cancer. In some cases, a cancer can be a metastatic cancer. In some cases a cancer can be a blood cancer. In some cases, a cancer can include one or more solid tumors. In some cases, a cancer can include one or more neuroendocrine tumors. Examples of cancers that can be associated with ANNA3 (e.g., can include the presence of ANNA3 autoantibodies in the blood of a mammal having cancer) include, without limitation, lung cancers (e.g., small cell lung cancers (SCLCs)), bladder cancers, teratomas, ovarian cancers, Merkel cell cancers, gastric cancers, and colon cancers, breast cancers.

Any appropriate sample from a mammal (e.g., human) having a neurologic autoimmune disorder (e.g., a paraneoplastic neurologic autoimmune disorder) and/or cancer can be assessed for the presence or absence of ANNA3 antibodies (e.g., ANNA3 autoantibodies). Examples of samples that can be assessed as described herein include, without limitation, blood samples (e.g., whole blood samples, serum samples, and plasma samples), and CSF samples. A sample can be a fresh sample or a fixed sample (e.g., a formaldehyde-fixed sample or a formalin-fixed sample). In some cases, one or more biological molecules can be isolated from a sample. For example, polypeptides (e.g., antibodies) can be isolated from a sample and can be assessed as described herein.

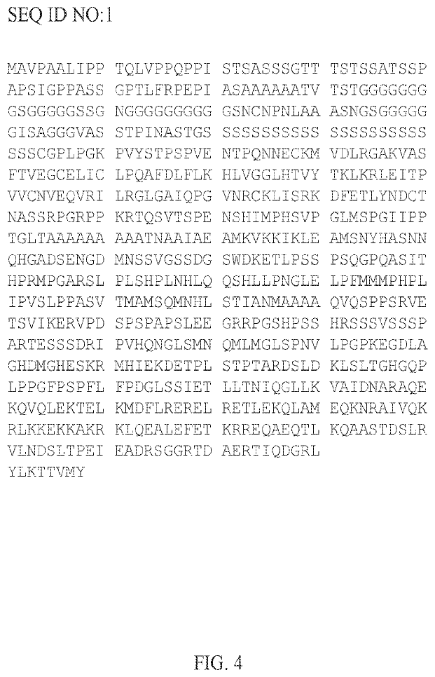

Any appropriate DACH1 polypeptide can be used to detect ANNA3 autoantibodies (e.g., in sample obtained from a mammal such as a human having a neurologic autoimmune disorder and/or cancer). Examples of DACH1 polypeptides that can be used to detect ANNA3 antibodies (e.g., ANNA3 autoantibodies) include those set forth in the National Center for Biotechnology Information (NCBI) databases at, for example, accession no. Q9UI36 (version Q9UI36.4), accession no. Q9QYB2 (version Q9QYB2.1), accession no. NP_004383 (version no. NP_004383.4), and accession no. NP_001353641 (version NP_001353641.1). In some cases, a DACH1 polypeptide that can be used to detect ANNA3 antibodies (e.g., ANNA3 autoantibodies) can have an amino acid sequence set forth in SEQ ID NO:1 (see, e.g., FIG. 4).

Any appropriate fragment of a DACH1 polypeptide can be used to detect ANNA3 antibodies. A fragment of a DACH1 polypeptide can be any appropriate length (e.g., can include any number of amino acids) provided that it retains the ability to bind to an ANNA3 antibody. For example, a fragment of a DACH1 polypeptide can be from about 9 amino acids in length to about 757 amino acids in length (e.g., from about 9 to about 700, from about 9 to about 650, from about 9 to about 600, from about 9 to about 550, from about 9 to about 500, from about 9 to about 450, from about 9 to about 400, from about 9 to about 350, from about 9 to about 300, from about 9 to about 250, from about 9 to about 200, from about 9 to about 150, from about 9 to about 100, from about 9 to about 50, from about 9 to about 25, from about 25 to about 757, from about 50 to about 757, from about 100 to about 757, from about 150 to about 757, from about 200 to about 757, from about 250 to about 757, from about 300 to about 757, from about 350 to about 757, from about 400 to about 757, from about 450 to about 757, from about 500 to about 757, from about 550 to about 757, from about 600 to about 757, from about 650 to about 757, from about 700 to about 757, from about 25 to about 700, from about 50 to about 700, from about 100 to about 600, from about 200 to about 500, from about 300 to about 400, from about 25 to about 100, from about 100 to about 200, from about 200 to about 300, from about 400 to about 500, or from about 500 to about 600 amino acids in length). In some cases, a fragment of a DACH1 polypeptide fragment can have the amino acid sequence set forth in SEQ ID NO:2 (e.g., can consist of the amino acid sequence set forth in SEQ ID NO:2). In some cases, a fragment of a DACH1 polypeptide can be a variant of the amino acid sequence set forth in SEQ ID NO:2 (e.g., can comprise or consist essentially of the amino acid sequence set forth in SEQ ID NO:2). For example, a variant of a fragment of a DACH1 polypeptide can consist of the amino acid sequence set forth in SEQ ID NO:2, except that the variant polypeptide includes one, two, three, four, five, or more amino acid substitutions within the articulated sequence of the sequence identifier (e.g., SEQ ID NO:2), has one, two, three, four, five, or more amino acid residues preceding the articulated sequence of the sequence identifier (e.g., SEQ ID NO:2), and/or has one, two, three, four, five, or more amino acid residues following the articulated sequence of the sequence identifier (e.g., SEQ ID NO:2), provided that the fragment of a DACH1 polypeptide retains the ability to bind to an ANNA3 antibody. Examples of fragments of a DACH1 polypeptide that can be used to detect ANNA3 antibodies include, without limitation, those fragments of a DACH1 polypeptide shown in Table 1.

TABLE 1
Examples of DACH1 polypeptides.

| Amino Acid Sequence | SEQ ID NO |
|---|---|
| ASSRPGRPPKRTQSVTSPENSHIMPHSVPGLMSPGIIPPTGLTAAAAAAAAATN | 2 |
| AAIAEAMKVKKIKLEAMSNYHASNNQHGADSENGDMNSSVGSSDGSWDKETLPS | |
| SPSQGPQASITHPRMPGARSLPLSHPLNHLQQSHLLPNGLELPFMMMPHPLIPV | |
| SLPPASVTMAMSQMNHLSTIANMAAAAQVQSPPSRVETSVIKERVPDSPSPAPS | |
| LEEGRRPGSHPSSHRSSSVSSSPARTESSSDRIPVHQNGLSMNQMLMGLSPNVL | |
| PGPKEGDLAGHDMGHESKRMHIEKDETPLSTPTARDSLDKLSLTGHGQPLPPGE | |
| PSPFLFPDGLSSIETLLTNIQGLLKVAIDNARAQEKQVQLEKTELKMDELRERE | |
| LRETLEKQLAMEQKNRAIVQKRLKKEKKAKRKLQEALEFETKRREQAEQTLKQA | |
| ASTDSLRVLNDSLTPEIEADRSGGRTDAERTIQDGRLYLKTTVMY | |
| MAVPAALIPPTQLVPPQPPISTSASSSGTTTSTSSATSSPAPSIGPPASSGPTL | 3 |
| FRPEPIASAAAAAATVTSTGGGGGGGGSGGGGGSSGNGGGGGGGGGGSNCNPNL | |
| AAASNGSGGGGGGISAGGGVASSTPINASTGSSSSSSSSSSSSSSSSSSSSSSS | |
| SCGPLPGKPVYSTPSPVENTPQNNECKMVDLRGAKVASFTVEGCELICLPQAFD | |
| LFLKHLVGGLHTVYTKLKRLEITPVVCNVEQVRILRGLGAIQPGVNRCKLISRK | |
| DFETLYNDCTNASSRPGRPPKRTQSVTSPENSHIMPHSVPGLMSPGIIPPTGLT | |
| AAAAAAAAATNAAIAEAMKVKKIKLEAMSNYHASNNQHGADSENGDMNSSVGLE | |
| LPFMMMPHPLIPVSLPPASVTMAMSQMNHLSTIANMAAAAQVQSPPSRVETSVI | |
| KERVPDSPSPAPSLEEGRRPGSHPSSHRSSSVSSSPARTESSSDRIPVHQNGLS | |
| MNQMLMGLSPNVLPGPKEGDLAGHDMGHESKRMHIEKDETPLSTPTARDSLDKL | |
| SLTGHGQPLPPGFPSPFLFPDGLSSIETLLTNIQGLLKVAIDNARAQEKQVQLE | |
| KTELKMDELRERELRETLEKQLAMEQKNRAIVQKRLKKEKKAKRKLQEALEFET | |
| KRREQAEQTLKQAASTDSLRVLNDSLTPEIEADRSGGRTDAERTIQDGRLYLKT | |
| TVMY | |
| MAVPAALIPPTQLVPPQPPISTSASSSGTTTSTSSATSSPAPSIGPPASSGPTL | 4 |
| FRPEPIASAAAAAATVTSTGGGGGGGGSGGGGGSSGNGGGGGGGGGGSNCNPNL | |
| AAASNGSGGGGGGISAGGGVASSTPINASTGSSSSSSSSSSSSSSSSSSSSSSS | |
| SCGPLPGKPVYSTPSPVENTPQNNECKMVDLRGAKVASFTVEGCELICLPQAFD | |
| LFLKHLVGGLHTVYTKLKRLEITPVVCNVEQVRILRGLGAIQPGVNRCKLISRK | |
| DFETLYNDCTNASSRPGRPPKRTQSVTSPENSHIMPHSVPGLMSPGIIPPTGLT | |
| AAAAAAAAATNAAIAEAMKVKKIKLEAMSNYHASNNQHGADSENGDMNSSVDET | |
| PLSTPTARDSLDKLSLTGHGQPLPPGFPSPFLEPDGLSSIETLLTNIQGLLKVA | |
| IDNARAQEKQVQLEKTELKMDELRERELRETLEKQLAMEQKNRAIVQKRLKKEK | |
| KAKRKLQEALEFETKRREQAEQTLKQAASTDSLRVLNDSLTPEIEADRSGGRTD | |
| AERTIQDGRLYLKTTVMY | |
| MAVPAALIPPTQLVPPQPPISTSASSSGTTTSTSSATSSPAPSIGPPASSGPTL | 5 |
| FRPEPIASAAAAAATVTSTGGGGGGGGSGGGGGSSGNGGGGGGGGGGSNCNPNL | |
| AAASNGSGGGGGGISAGGGVASSTPINASTGSSSSSSSSSSSSSSSSSSSSSSS | |
| SCGPLPGKPVYSTPSPVENTPQNNECKMVDLRGAKVASFTVEGCELICLPQAFD | |
| LFLKHLVGGLHTVYTKLKRLEITPVVCNVEQVRILRGLGAIQPGVNRCKLISRK | |
| DFETLYNDCTNASSRPGRPPKRTQSVTSPENSHIMPHSVPGLMSPGIIPPTDET | |
| PLSTPTARDSLDKLSLTGHGQPLPPGFPSPFLFPDGLSSIETLLTNIQGLLKVA | |
| IDNARAQEKQVQLEKTELKMDELRERELRETLEKQLAMEQKNRAIVQKRLKKEK | |
| KAKRKLQEALEFETKRREQAEQTLKQAASTDSLRVLNDSLTPEIEADRSGGRTD | |
| AERTIQDGRLYLKTTVMY | |
| QAASTDSLRVLNDSLTPEIEADRSGGRTDAERTIQDGRLYLKTTVMY | 6 |
| ASSRPGRPPKRTQSVT | 7 |

Any appropriate method can be used to obtain a DACH1 polypeptide or a fragment of a DACH1 polypeptide described herein (e.g., a polypeptide that consists of the amino acid sequence set forth in SEQ ID NO:1 or a variant thereof). In some cases, a DACH1 polypeptide or a fragment of a DACH1 polypeptide can be obtained using polypeptide synthesizing methods. For example, a polynucleotide sequence encoding a DACH1 polypeptide and/or a polynucleotide sequence encoding a fragment of a DACH1 polypeptide can be inserted into a plasmid or other vector that can then be delivered to host cells that can be induced to transcribe and translate the polynucleotide into the polypeptide. In some cases, a polynucleotide sequence for a larger polypeptide (e.g., a DACH1 polypeptide) can be inserted into host cells that can produce the larger polypeptide and then process that polypeptide into a smaller polypeptide or a functional variant of interest (e.g., a fragment of a DACH1 polypeptide).

A DACH1 polypeptide and/or one or more fragments of a DACH1 polypeptide can be provided in any appropriate context. In some cases, a DACH1 polypeptide and/or one or more fragments of a DACH1 polypeptide that can be used to detect the presence or absence of ANNA3 antibodies (e.g., ANNA3 autoantibodies) can be present in a cell lysate. For example, a DACH1 polypeptide can be present in a cell lysate obtained from a cell having (e.g., designed to have) an exogenous nucleic acid encoding the DACH1 polypeptide. For example, a fragment of a DACH1 polypeptide can be present in a cell lysate obtained from a cell having (e.g., designed to have) an exogenous nucleic acid encoding the fragment of a DACH1 polypeptide.

In some cases, a DACH1 polypeptide and/or one or more fragments of a DACH1 polypeptide that can be used to detect the presence or absence of ANNA3 antibodies (e.g., ANNA3 autoantibodies) can be present on a cell (e.g., an intact cell). For example, a DACH polypeptide can be present on a cell having (e.g., designed to have) an exogenous nucleic acid encoding the DACH1 polypeptide and expressing the DACH1 polypeptide on its surface. For example, a fragment of a DACH polypeptide can be present on a cell having (e.g., designed to have) an exogenous nucleic acid encoding the fragment of a DACH1 polypeptide and expressing the fragment on its surface.

In some cases, a DACH1 polypeptide and/or one or more fragments of a DACH1 polypeptide that can be used to detect the presence or absence of ANNA3 antibodies (e.g., ANNA3 autoantibodies) can be substantially pure. The term “substantially pure” as used herein with reference to a DACH1 polypeptide and/or one or more fragments of a DACH1 polypeptide refers to material which is substantially or essentially free from components (e.g., other polypeptides, lipids, carbohydrates, and nucleic acid) that normally accompany the material as it is found in its native state. Thus, substantially pure polypeptides as described in this document do not contain at least some of the materials normally associated with the polypeptides in their in situ environment. For example, a substantially pure DACH1 polypeptide and/or one or more substantially pure fragments of a DACH1 polypeptide can constitute the major component in a mixture of components (e.g., 30% or more, 40% or more, 50% or more, 60% or more, 70% or more, 80% or more, 90% or more, 95% or more, or 99% or more by weight).

In some cases, a DACH1 polypeptide and/or one or more fragments of a DACH1 polypeptide that can be used to detect the presence or absence of ANNA3 antibodies (e.g., ANNA3 autoantibodies) can be present in a composition. A composition including a DACH1 polypeptide and/or one or more fragments of a DACH1 polypeptide that can be used to detect the presence or absence of ANNA3 antibodies (e.g., ANNA3 autoantibodies) can include any appropriate amount of the DACH1 polypeptide and/or the one or more fragments of a DACH1 polypeptide. For example, at least 5 percent (e.g., at least 10%, at least 20%, at least 30%, at least 40%, at least 50%, at least 60%, at least 70%, at least 80%, at least 90%, or more) of the polypeptide content of a composition including a DACH1 polypeptide and/or one or more fragments of a DACH1 polypeptide can be the DACH1 polypeptide and/or the one or more fragments of a DACH1 polypeptide. In some cases, a composition including a DACH1 polypeptide and/or one or more fragments of a DACH1 polypeptide can be enriched for the DACH1 polypeptide and/or the one or more fragments of a DACH1 polypeptide.

In some cases, a DACH1 polypeptide and/or one or more fragments of a DACH1 polypeptide described herein can lack any modification. For example, a DACH1 polypeptide and/or one or more fragments of a DACH1 polypeptide can be used to detect the presence or absence of ANNA3 antibodies (e.g., ANNA3 autoantibodies) without any modification. For example, a DACH1 polypeptide and/or one or more fragments of a DACH1 polypeptide that can be used to detect the presence or absence of ANNA3 antibodies (e.g., ANNA3 autoantibodies) can lack any detectable label.

In some cases, a DACH1 polypeptide and/or one or more fragments of a DACH1 polypeptide described herein can be modified. For example, a DACH1 polypeptide and/or one or more fragments of a DACH1 polypeptide that can be used for detecting the presence or absence of ANNA3 antibodies (e.g., ANNA3 autoantibodies) can include (e.g., can be covalently linked to) one or more labels (e.g., one or more detectable labels). In some cases, a label can be a polypeptide tag (e.g., an affinity tag). In some cases, a label can be a fluorescent label. In some cases, a label can be covalently linked to a chemiluminescent label. In some cases, a label can have enzymatic activity. In some cases, a label can be radioactive. Examples of labels that can be attached to DACH1 polypeptide or a fragment of a DACH1 polypeptide include, without limitation, tetramethylrhodamine isothiocyanate (TRITC), fluorescein isothiocyanate (FITC), poly(His) tags, and glutathione-S-transferase (GST) tags.

Any appropriate method can be used to detect the presence or absence of ANNA3 antibodies (e.g., ANNA3 autoantibodies) in a sample (e.g., a serum sample) obtained from a mammal (e.g., a human) having a neurologic autoimmune disorder (e.g., a paraneoplastic neurologic autoimmune disorder) and/or cancer. For example, immunological assays using a DACH1 polypeptide and/or one or more fragments of a DACH1 polypeptide provided herein can be used to determine if a sample contains ANNA3 autoantibodies. In some cases, a sample (e.g., a serum sample) obtained from a mammal (e.g., a human) having a neurologic autoimmune disorder (e.g., a paraneoplastic neurologic autoimmune disorder) and/or cancer can be contacted with a DACH1 polypeptide and/or one or more fragments of a DACH1 polypeptide described herein such that an ANNA3 antibody, if present, forms a complex with the DACH1 polypeptide and/or the one or more fragments of a DACH1 polypeptide, and the presence or absence of a complex can be used to determine whether or not ANNA3 autoantibodies are present in the sample. In some cases, an immobilized DACH1 polypeptide and/or one or more immobilized fragments of a DACH1 polypeptide can be used to capture an ANNA3 antibody if present within a sample being tested, and secondary antibody such as an anti-Ig antibody (e.g., an anti-human IgG antibody when testing for human autoantibodies) can be used to determine whether or not ANNA3 autoantibodies were captured. In some cases, an anti-Ig antibody can be labeled (e.g., fluorescently or enzymatically labeled) to aid in detection.

In some cases, the presence or absence of ANNA3 antibodies (e.g., ANNA3 autoantibodies) in a sample (e.g., a serum sample) obtained from a mammal (e.g., a human) having a neurologic autoimmune disorder (e.g., a paraneoplastic neurologic autoimmune disorder) and/or cancer can be detected as described in Example 1.

In some cases, methods for assessing a sample (e.g., a serum sample) obtained from a mammal (e.g., a human) having a neurologic autoimmune disorder (e.g., a paraneoplastic neurologic autoimmune disorder) for the presence or absence of ANNA3 antibodies (e.g., ANNA3 autoantibodies) in a sample (e.g., a serum sample) obtained from the mammal as provided herein can be used to identify a mammal having an ANNA3-positive neurologic autoimmune disorder. For example, when a mammal (e.g., a human) having a neurologic autoimmune disorder (e.g., a paraneoplastic neurologic autoimmune disorder) is identified as having a presence of ANNA3 antibodies (e.g., ANNA3 autoantibodies) in a sample (e.g., a serum sample) obtained from the mammal, the mammal can be classified as having an ANNA3-positive neurologic autoimmune disorder. For example, when a mammal (e.g., a human) having a neurological autoimmune disorder (e.g., a paraneoplastic neurological autoimmune disorder) is identified as lacking ANNA3 antibodies (e.g., ANNA3 autoantibodies) in a sample (e.g., a serum sample) obtained from the mammal, the mammal can be classified as not having an ANNA3-positive neurologic autoimmune disorder. For example, when a mammal (e.g., a human) having a neurological autoimmune disorder (e.g., a paraneoplastic neurological autoimmune disorder) is identified as lacking ANNA3 antibodies (e.g., ANNA3 autoantibodies) in a sample (e.g., a serum sample) obtained from the mammal, the mammal can be classified as having an ANNA3-negative neurologic autoimmune disorder.

In some cases, methods for assessing a sample (e.g., a serum sample) obtained from a mammal (e.g., a human) having cancer for the presence or absence of ANNA3 antibodies (e.g., ANNA3 autoantibodies) in a sample (e.g., a serum sample) obtained from the mammal as provided herein can be used to identify a mammal having an ANNA3-positive cancer. For example, when a mammal (e.g., a human) having cancer is identified as having a presence of ANNA3 antibodies (e.g., ANNA3 autoantibodies) in a sample (e.g., a serum sample) obtained from the mammal, the mammal can be classified as having an ANNA3-positive cancer. For example, when a mammal (e.g., a human) having cancer is identified as lacking ANNA3 antibodies (e.g., ANNA3 autoantibodies) in a sample (e.g., a serum sample) obtained from the mammal, the mammal can be classified as not having an ANNA3-positive cancer. For example, when a mammal (e.g., a human) having cancer is identified as lacking ANNA3 antibodies (e.g., ANNA3 autoantibodies) in a sample (e.g., a serum sample) obtained from the mammal, the mammal can be classified as having an ANNA3-negative cancer.

In some cases, when a mammal (e.g., a human) having a neurologic autoimmune disorder (e.g., a paraneoplastic neurologic autoimmune disorder) is identified as having an ANNA3-positive neurologic autoimmune disorder as described herein (e.g., based, at least in part, on the presence of ANNA3 antibodies in a sample obtained from a mammal), the mammal can be assessed to determine whether or not the mammal has cancer. Any appropriate method can be used to identify a mammal as having cancer.

This document also provides methods and materials for treating a mammal (e.g., a human) having a neurologic autoimmune disorder (e.g., a paraneoplastic neurologic autoimmune disorder) and/or cancer, where one or more treatments are selected based on whether the mammal is identified as having an ANNA3-positive neurologic autoimmune disorder and/or an ANNA3-positive cancer as described herein (e.g., based, at least in part, on the presence or absence of ANNA3 antibodies in a sample obtained from a mammal). For example, a sample (e.g., a serum sample) obtained from a mammal (e.g., a mammal such as a human having a neurologic autoimmune disorder and/or cancer) can be assessed for the presence or absence of ANNA3 antibodies (e.g., ANNA3 autoantibodies), and one or more treatments can be selected and, optionally, administered, to the mammal based, at least in part, on whether the presence or absence of ANNA3 antibodies (e.g., ANNA3 autoantibodies) is detected.

In some cases, a DACH1 polypeptide and/or one or more fragments of a DACH1 polypeptide described herein can be used in an apheresis method to treat a mammal (e.g., a human) having a neurologic autoimmune disorder (e.g., a paraneoplastic neurologic autoimmune disorder) and/or cancer. For example, a DACH1 polypeptide and/or one or more fragments of a DACH1 polypeptide described herein can be used in an apheresis for the treatment of a neurologic autoimmune disorder (e.g., a paraneoplastic neurologic autoimmune disorders) and/or cancer associated with ANNA3 autoantibodies to remove ANNA3 autoantibodies from the mammal. In some cases, an apheresis method to remove ANNA3 autoantibodies from a blood of a mammal (e.g., a human such as a human having a neurologic autoimmune disorder and/or cancer) can include withdrawing blood from the mammal; contacting the blood with a DACH1 polypeptide and/or one or more fragments of a DACH1 polypeptide described herein to remove a substantial portion of ANNA3 autoantibodies from the blood; and returning the blood to the mammal. In some cases, methods and extracorporeal systems for apheresis (i.e., the process of withdrawing blood from an individual, removing components from the blood, and returning the blood, or blood depleted of one or more components, to the individual) can be used as described elsewhere (see, for example, Lehmann et al. (Arch Neurol. 63(7):930-5 (2006)); and U.S. Pat. Nos. 4,708,713; 5,258,503; 5,386,734; and 6,409,696). As used herein, a “substantial portion” means removing at least 20% (e.g., at least: 20%; 30%; 40%; 50%; 60%; 65%; 70%; 75%; 80%; 85%; 90%; 93%; 95%; 96%; 97%; 98%; 99%; 99.5%; 99.8%; or even 100%) of the ANNA3 autoantibodies that were present in the blood prior to removal.

When a mammal (e.g., a human) having a neurologic autoimmune disorder (e.g., a paraneoplastic neurologic autoimmune disorder) is identified as having an ANNA3-positive neurologic autoimmune disorder as described herein (e.g., based, at least in part, on the presence of ANNA3 antibodies in a sample obtained from a mammal), the mammal can be administered, or instructed to self-administer, one or more therapies effective to treat an ANNA3-positive neurologic autoimmune disorder (e.g., an ANNA3-positive paraneoplastic neurologic autoimmune disorder). In some cases, a therapy that can be effective to treat an ANNA3-positive neurologic autoimmune disorder (e.g., an ANNA3-positive paraneoplastic neurologic autoimmune disorder) can include plasma exchange therapy (plasmapheresis). In some cases, a therapy that can be effective to treat an ANNA3-positive neurologic autoimmune disorder (e.g., an ANNA3-positive paraneoplastic neurologic autoimmune disorder) can include intravenous immunoglobulin therapy. In some cases, a therapy that can be effective to treat an ANNA3-positive neurologic autoimmune disorder (e.g., an ANNA3-positive paraneoplastic neurologic autoimmune disorder) can include administering one or more agents that can be effective to treat an ANNA3-positive neurologic autoimmune disorder (e.g., an ANNA3-positive paraneoplastic neurologic autoimmune disorder). In some cases, an agent that can be effective to treat an ANNA3-positive neurologic autoimmune disorder (e.g., an ANNA3-positive paraneoplastic neurologic autoimmune disorder) can be an immunosuppressant. In some cases, an agent that can be effective to treat an ANNA3-positive neurologic autoimmune disorder (e.g., an ANNA3-positive paraneoplastic neurologic autoimmune disorder) can be an anti-inflammatory agent. Examples of agents that can be used to treat a mammal identified as having ANNA3-positive neurologic autoimmune disorder as described herein (e.g., based, at least in part, on the presence of ANNA3 antibodies in a sample obtained from a mammal) include, without limitation, cyclophosphamide, rituximab, azathioprine, mycophenolate mofetil, methotrexate, and steroids (e.g., corticosteroids).

In some cases, when a mammal (e.g., a human) having a neurologic autoimmune disorder (e.g., a paraneoplastic neurologic autoimmune disorder) is identified as having an ANNA3-positive neurologic autoimmune disorder as described herein (e.g., based, at least in part, on the presence of ANNA3 antibodies in a sample obtained from a mammal), the mammal is not administered, or is instructed to not self-administer, any therapy typically used to treat psychiatric disorders (e.g., primary psychiatric disorders), degenerative diseases (e.g., neurodegenerative diseases of the central nervous system and/or peripheral nervous system) and/or brain cancer (e.g., cancer that as metastasized to the brain). Examples of therapies that are typically used to treat psychiatric disorders (e.g., primary psychiatric disorders), degenerative diseases (e.g., neurodegenerative diseases of the central nervous system and/or peripheral nervous system) and/or brain cancer (e.g., cancer that as metastasized to the brain) include, without limitation, donepezil, memantine, riluzole, edaravone, haloperidol, and quetiapine.

In some cases, when treating a mammal (e.g., a human) having a neurologic autoimmune disorder (e.g., a paraneoplastic neurologic autoimmune disorder) as described herein, the treatment can be effective to reduce or eliminate one or more symptoms of the neurologic autoimmune disorder. Examples of symptoms of neurologic autoimmune disorders include, without limitation, dysautonomia, ataxia (e.g., cerebellar ataxia and sensory ataxia), neuropathy (e.g., small fiber neuropathy, motor neuropathy, sensory neuropathy, sensorimotor neuropathy, optic neuropathy, and polyradiculoneuropathy), cognitive decline, encephalitis (e.g., limbic encephalitis), seizures, encephalopathy (e.g., subacute brainstem encephalopathy), eye movement disorder with diplopia, ptosis, dysphagia, dementia, weakness (e.g., facial weakness), chorioretinopathy, dizziness, parkinsonism, myelopathy, hyponatremia, vertigo, and urinary retention. For example, the methods and materials described herein can be used to reduce one or more symptoms within a mammal having a neurologic autoimmune disorder (e.g., a paraneoplastic neurologic autoimmune disorder) by, for example, 10, 20, 30, 40, 50, 60, 70, 80, 90, 95, or more percent.

In some cases, when treating a mammal (e.g., a human) having a neurologic autoimmune disorder (e.g., a paraneoplastic neurologic autoimmune disorder) as described herein, the treatment can be effective to reduce or eliminate inflammation within the mammal (e.g., within the mammal's nervous system). For example, the methods and materials described herein can be used to reduce inflammation within a mammal having a neurologic autoimmune disorder (e.g., a paraneoplastic neurologic autoimmune disorder) by, for example, 10, 20, 30, 40, 50, 60, 70, 80, 90, 95, or more percent.

In some cases, when treating a mammal (e.g., a human) having a neurologic autoimmune disorder (e.g., a paraneoplastic neurologic autoimmune disorder) as described herein, the treatment can be effective to reduce or eliminate the number of ANNA3 antibodies (e.g., ANNA3 autoantibodies) within the mammal. For example, the number of ANNA3 antibodies (e.g., ANNA3 autoantibodies) present within a mammal can be reduced using the methods and materials described herein. In some cases, the methods and materials described herein can be used to reduce the number of ANNA3 antibodies (e.g., ANNA3 autoantibodies) present within a mammal having cancer by, for example, 10, 20, 30, 40, 50, 60, 70, 80, 90, 95, or more percent. In some cases, the number of ANNA3 antibodies (e.g., ANNA3 autoantibodies) present within a mammal does not increase.

When a mammal (e.g., a human) having cancer is identified as having ANNA3-positive cancer as described herein (e.g., based, at least in part, on the presence of ANNA3 antibodies in a sample obtained from a mammal), the mammal can be administered, or instructed to self-administer, one or more therapies effective to treat the cancer. In some cases, a therapy that can be effective to treat cancer can include radiation therapy. In some cases, a therapy that can be effective to treat cancer can include surgery. In some cases, a therapy that can be effective to treat cancer can include administering one or more agents that can be effective to treat cancer (e.g., administering one or more anti-cancer agents).

In some cases, when a mammal (e.g., a human) having cancer is identified as having ANNA3-positive cancer as described herein (e.g., based, at least in part, on the presence of ANNA3 antibodies in a sample obtained from a mammal), the mammal is not administered, or is instructed to not self-administer, any immunotherapy typically used to treat cancer. In some cases, an immunotherapy can include one or more immune checkpoint inhibitors such as inhibitors of a CTLA4 polypeptide, inhibitors of a PD-1 polypeptide, and inhibitors of a PD-L1 polypeptide. Examples of immunotherapies that are typically used to treat cancer include, without limitation, ipilimumab, nivolumab, pembrolizumab, atezolizumab, avelumab, durvalumab, and cemiplimab.

When a mammal (e.g., a human) having cancer is identified as having ANNA3-negative cancer as described herein (e.g., based, at least in part, on the absence of ANNA3 antibodies in a sample obtained from a mammal), the mammal can be administered, or instructed to self-administer, one or more therapies effective to treat ANNA3-negative cancer. In some cases, a therapy that can be effective to treat an ANNA3-negative cancer can include radiation therapy. In some cases, a therapy that can be effective to treat an ANNA3-negative cancer can include surgery. In some cases, a therapy that can be effective to treat an ANNA3-negative cancer can include administering one or more agents that can be effective to treat an ANNA3-negative cancer (e.g., administering one or more anti-cancer agents). In some cases, a therapy that can be effective to treat an ANNA3-negative cancer can include administering one or more immunotherapies (e.g., administering one or more immune checkpoint inhibitors such as inhibitors of a CTLA4 polypeptide, inhibitors of a PD-1 polypeptide, and inhibitors of a PD-L1 polypeptide). Examples of agents that can be used to treat a mammal identified as having ANNA3-negative cancer as described herein (e.g., based, at least in part, on the presence of ANNA3 antibodies in a sample obtained from a mammal) include, without limitation, ipilimumab, nivolumab, pembrolizumab, atezolizumab, avelumab, durvalumab, and cemiplimab.

In some cases, when treating a mammal (e.g., a human) having cancer as described herein, the treatment can be effective to treat the cancer. For example, the number of cancer cells present within a mammal can be reduced using the methods and materials described herein. In some cases, the methods and materials described herein can be used to reduce the number of cancer cells present within a mammal having cancer by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having cancer by, for example, 10, 20, 30, 40, 50, 60, 70, 80, 90, 95, or more percent. In some cases, the size (e.g., volume) of one or more tumors present within a mammal does not increase.

In some cases, when a mammal (e.g., a human) having a neurologic autoimmune disorder (e.g., a paraneoplastic neurologic autoimmune disorder) is identified as having an ANNA3-positive neurologic autoimmune disorder as described herein (e.g., based, at least in part, on the presence of ANNA3 antibodies in a sample obtained from a mammal) and as having cancer, the mammal can be administered, or instructed to self-administer, one or more therapies effective to treat the cancer. In some cases, a therapy that can be effective to treat cancer can include radiation therapy. In some cases, a therapy that can be effective to treat cancer can include surgery. In some cases, a therapy that can be effective to treat cancer can include administering one or more agents that can be effective to treat cancer (e.g., administering one or more anti-cancer agents). In some cases, a therapy that can be effective to treat cancer can be effective to treat both the neurologic autoimmune disorder and the cancer.

In some cases, when treating a mammal (e.g., a human) having an ANNA3-positive neurologic autoimmune disorder and having cancer as described herein, the mammal is not administered, or is instructed to not self-administer, any immunotherapy typically used to treat cancer. In some cases, an immunotherapy can include one or more immune checkpoint inhibitors such as inhibitors of a CTLA4 polypeptide, inhibitors of a PD-1 polypeptide, and inhibitors of a PD-L1 polypeptide. Examples of immunotherapies that are typically used to treat cancer include, without limitation, ipilimumab, nivolumab, pembrolizumab, atezolizumab, avelumab, durvalumab, and cemiplimab.

In some cases, methods for assessing a sample (e.g., a serum sample) obtained from a mammal (e.g., a human) having a neurologic autoimmune disorder (e.g., a paraneoplastic neurologic autoimmune disorder) and/or cancer for the presence or absence of ANNA3 antibodies (e.g., ANNA3 autoantibodies) can be used to monitor a course of treatment. In some cases, a mammal being treated for a neurologic autoimmune disorder (e.g., a paraneoplastic neurologic autoimmune disorder) and identified as having an ANNA3-positive neurologic autoimmune disorder as described herein (e.g., based, at least in part, on the presence of ANNA3 antibodies in a sample obtained from a mammal) can be monitored for the presence, absence, or level of ANNA3 antibodies during treatment.

The invention will be further described in the following examples, which do not limit the scope of the invention described in the claims.

### EXAMPLES

### Example 1: Anti-Neuronal Nuclear Antibody 3 Autoimmunity Targets Dachshund Homolog 1

This Example identifies Dachshund homolog 1 (DACH1) as the autoantigen for anti-neuronal nuclear antibody-type 3 (ANNA3)-IgG, and describes how DACH1 can be used as a serological biomarker of neurological autoimmunity and/or cancer.

### Patients

Patients were identified through a database. All patients in whom ANNA3-IgG was detected via service neural autoantibody testing were included. Residual serum or CSF specimens were available for 32 patients (26 archival and six identified prospectively during this study; 21 serum only, 3 CSF only and 8 both serum and CSF). Clinical information, available for 30 patients, was abstracted from electronic medical records described elsewhere (Dalmau et al., Medicine (Baltimore), 71:59-72 (1992)) or provided by referring physicians of patients as described elsewhere (Pittock et al., Arch. Neurol., 67:1109-1115 (2010)). Control patients (145 total) included 21 healthy subjects, 10 anti-nuclear antibody (ANA)-IgG seropositive patients, 30 patients with SCLC diagnosis regardless of antibody status, 60 patients positive for paraneoplastic neural autoantibodies specific for intracellular antigens (ANNA1-IgG, 34; ANNA2-IgG, 7; CRMP5-IgG, 12 or amphiphysin-IgG, 7), and 24 positive for neural autoantibodies specific for cell-surface antigens (AQP4-IgG;12 and LGI1-IgG;12).

### Laboratory Methods

### Murine Tissue Indirect Immunofluorescence

Patients' specimens were screened at 1:240 dilution (serum; preabsorbed with bovine liver powder) or 1:2 (CSF) on a cryosectioned composite of murine brain, kidney and gut tissues (fixed for 10 minutes with 10% buffered formalin, then washed with phosphate-buffered saline (PBS), pH 7.4). DACH1-specific polyclonal antibody (PA552968, Thermo Fisher) was diluted 1:100. Normal goat serum (10% in PBS) was applied for 1 hour to block non-specific binding of secondary antibodies (goat anti-rabbit-IgG (FITC-conjugated) and goat anti-human IgG (TRITC-conjugated or FITC-conjugated), Southern Biotech; all 1:200 dilution). After 40 minutes in primary antibody, PBS wash, and 30 minutes in secondary antibody, slides were washed and mounted (ProLong Diamond Antifade Mountant, Life Sciences, P36962). All samples were read blinded by two experienced readers. Positives were titrated in doubling steps to determine positivity endpoint.

### SCLC Cell-Line Immunoreactivity

Sixteen ANNA3-IgG-positive patients' sera were tested by indirect immunofluorescence (1:100 and 1:800 dilution), on SCC37 cells (SCLC line established in the Neuroimmunology Laboratory), fixed (4% PFA), permeabilized (0.2% Triton X-100) and blocked with 10% normal goat serum.

### Protein Characterization

### Nuclear Lysate Preparation

All steps were at 4° C. Up to 1×10⁹ SCC37 cells (minimum 2×10⁸) were used. Cells were lifted with trypsin (0.25%), washed with cold PBS were resuspended in five times the estimated packed cell volume (pcv) in hypotonic buffer (10 mM HEPES, pH 7.9, 1.5 mM MgCl₂, 10 mM KCl, 0.5 M DTT, protease inhibitor tablets (complete, Mini; Sigma-Aldrich, St. Louis, MO)). After centrifugation at 1850×g, the cells were resuspended in 3-times pcv hypotonic buffer. After swelling 10 minutes, cells were homogenized (glass dounce, type B pestle) and centrifuged (3300×g, 15 minutes). The pellet was resuspended in low-salt buffer (20 mM HEPES, pH 7.9. 25% glycerol, 1.5 mM, MgCl2, 0.5 M, DTT, protease inhibitor tablets). 5M NaCl was added slowly to 0.42M final concentration, and after 30 minutes gentle mixing, chromatin was pelleted by ultracentrifugation (100,000×g, 1 hour). Supernatant nuclear extract was stored at −80° C. Western blot was used to identify immunoreactive bands common to all ANNA3-IgG-positive patients amongst electrophoretically separated proteins (reducing/denaturing 4-15% polyacrylamide gel, Bio-Rad).

### Antibody Purification

IgGs isolated from sera of ANNA3-IgG-positive patients and healthy controls (by adsorption to protein G; Dynabeads, Invitrogen) were incubated with the SCC37 nuclear extract. IgG and bound proteins eluted and dissociated by boiling in sodium dodecyl sulfate sample (SDS) buffer, were separated by gel electrophoresis. Common immunoreactive bands, identified by silver staining and western blot, were excised, and proteins were identified by mass spectrometry.

To confirm that candidate immunoreactive bands contained the antigen yielding the ANNA3 immunohistochemical staining pattern, IgG was acid-eluted from excised nitrocellulose bands of corresponding molecular weight, as well as from an independent control section of nitrocellulose, and applied (after neutralization) to mouse tissue sections for immunofluorescence evaluation described elsewhere (Zekeridou et al., Neurology, 93(8):e815-e822 (2019)).

### DACH1 Antigen Confirmation

### Western Blot

Preparation of lysates from transfected cells overexpressing DACH1. HEK293 cells were homogenized in RIPA buffer and sonicated 48 hours after transfection with plasmid encoding full-length DACH1 protein. The 33000×g-clarified supernate was subjected to western blot analysis.

DACH1 polypeptide production. A gene fragment encoding the DACH1 polypeptide (residues 282-758) was cloned into a pET28 vector with an N-terminal 6×His-tag (Genscript) to transform E. coli (strain BL21-Codon plus (DE3)-RIPL; Agilent) which were grown in LB broth media containing kanamycin (50 μg/mL) and chloramphenicol (50 μg/mL). Recombinant protein synthesis was induced using Overnight Express Autoinduction System 1 (Novagen). Cells expressing DACH1-282-758 were lysed using BugBuster Protein Extraction Reagent (EMD Millipore). Washed inclusion bodies were solubilized in 8M guanidinium chloride (GdnHCl), and added to denaturing buffer (100 mM sodium phosphate, 10 mM Tris, 6 M guanidine, pH8.0). Proteins of interest were captured by Ni-NTA resin and eluted in denaturing buffer containing 250 mM imidazole after washing in buffer containing 20 mM of imidazole. Eluted protein was first dialyzed into storage buffer (70 mM Tris-HCl pH 8.0, 1 mM EDTA, 200 mM NaCl, 5% glycerol) containing 3M GdnHCl, then into storage buffer without GdnHCl. 6.9 μg of protein/gel was used for western blot.

Absorption of Patient Serum with DACH1 Protein

Sera from two ANNA3-IgG-positive patients and a control ANNA1-IgG-positive patient were incubated with DACH1-HEK293 cell lysate (1 hour at 4° C.) and tested by IFA on mouse cryosections.

### Cell-Based Assays (CBA)

HEK293 cells grown on glass coverslips were transiently transfected with plasmid encoding DACH1-C-EGFP (GenScript, NM_001366712.1), fixed with 4% PFA, permeabilized with 0.2% Triton X-100 and tested by indirect immunofluorescence with patient sera (at 1:50 and 1:500) or CSF (1:2). CBAs of both ANNA3-IgG-positive patients and controls were interpreted blinded by two experienced readers.

### Immunohistochemistry and Neuropathology

Heat-induced antigen retrieval was performed on deparaffinized PFA-fixed tumor sections (5 μm thick), (20 minutes; EnVision™ FLEX high pH target antigen retrieval solution (Dako)). Subsequent immunostaining used IgGs specific for DACH1 (1:100, Invitrogen), synaptophysin (1:200, Leica), chromogranin A (1:800, Invitrogen) and thyroid transcription factor (TTF-1, 1:200, DAKO) and the EnVision™ FLEX immunohistochemistry system (Dako). Stained sections were counterstained with hematoxylin (Dako).

### Results

### Antigen Characterization

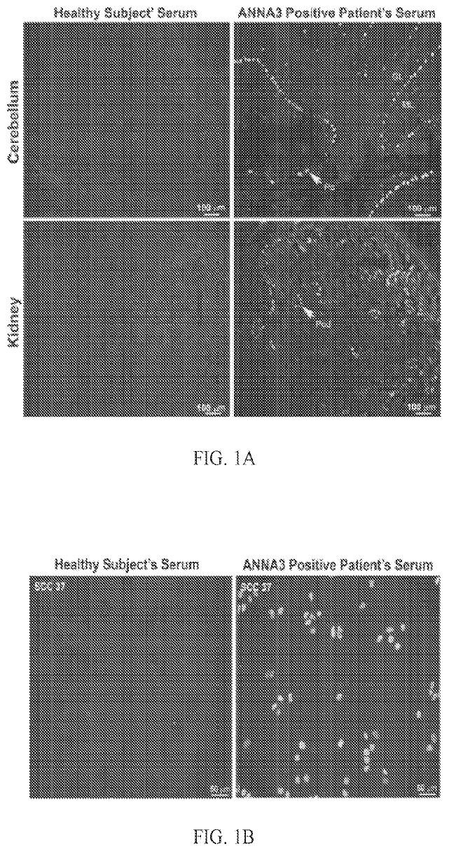

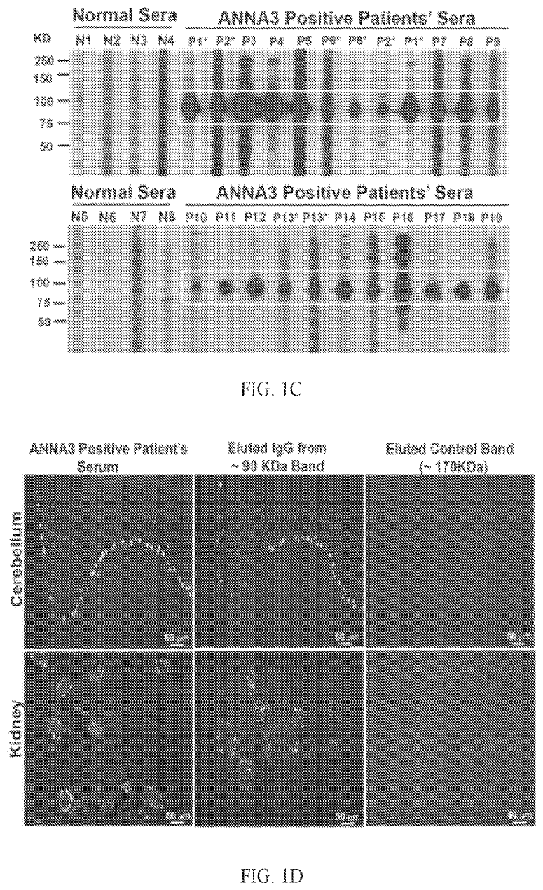

All patients' specimens yielded nuclear immunofluorescence staining typical of ANNA3-IgG binding to cerebellum (prominent in Purkinje neurons, fainter in molecular layer neurons) and renal cortex (prominent in podocytes; FIG. 1A). Immunoreactivity in SCLC tumor cells was confirmed immunohistochemically and by western blot (FIGS. 1B and 1C). Available ANNA3-IgG-positive sera (19 patients) bound to a protein smaller than 100 kDa. IgG eluted from the nitrocellulose-corresponding band yielded the same staining pattern as the original sera on mouse tissue sections (FIG. 1D), but IgG eluted from an irrelevant region of nitrocellulose did not. Mass spectrometric analysis revealed peptides derived from DACH1 (predicted MW 76 kDa) as most abundant amongst proteins captured from lysed SCC37 nuclei by ANNA3-positive patients' IgG (but not by control-IgG).

### Confirmation of DACH1 as ANNA3 Autoantigen

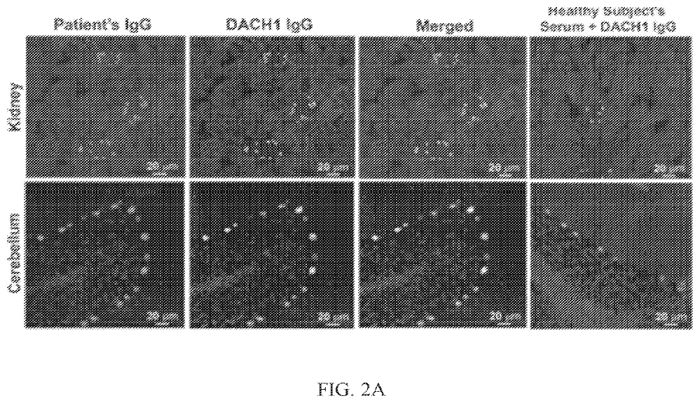

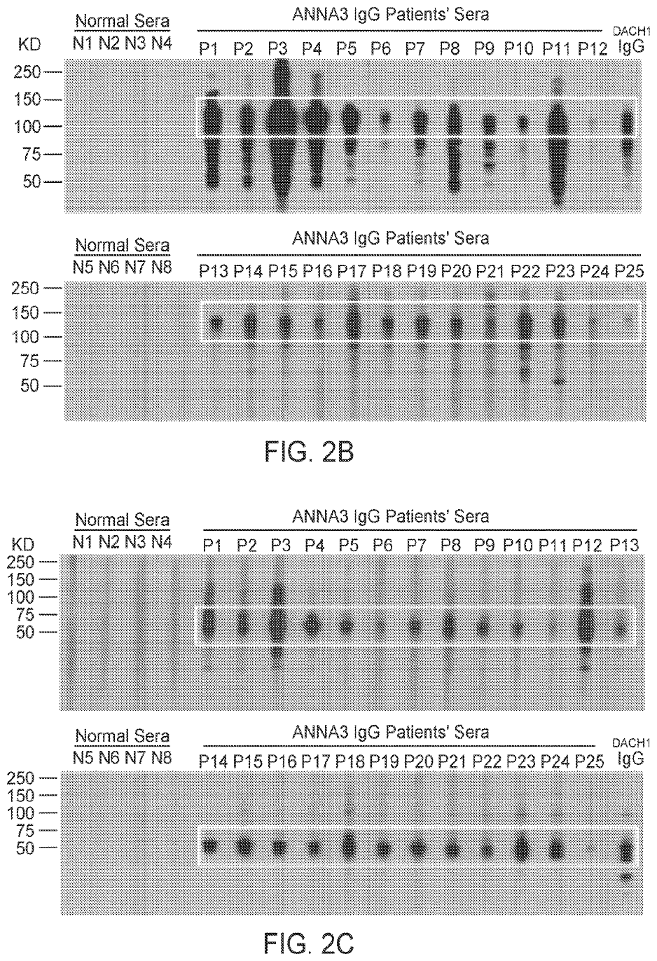

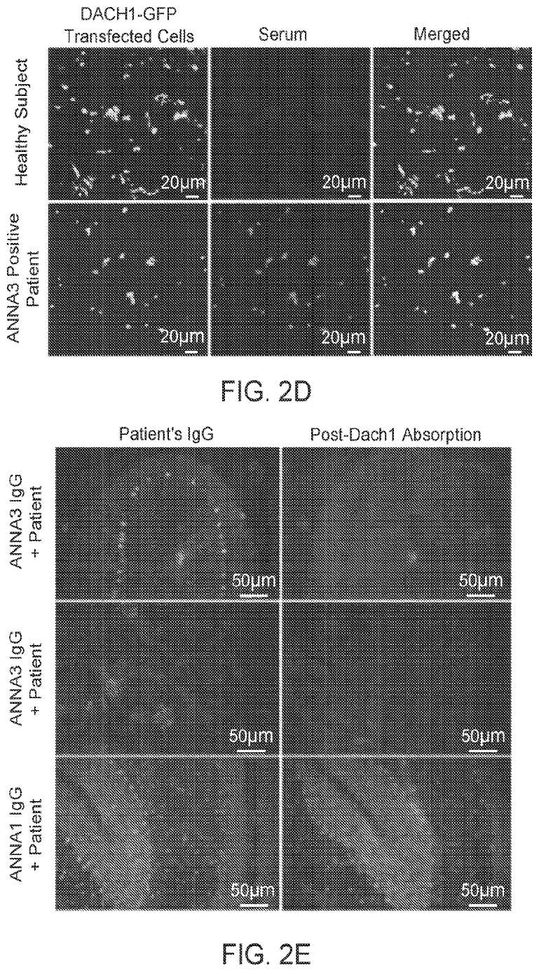

It was confirmed that DACH1, the immunocaptured protein identified by mass spectrometry, was the pertinent ANNA3 antigen as follows (FIG. 2): Firstly, IgG in ANNA3-positive sera colocalized with a commercial DACH1-specific IgG by confocal microscopy (FIG. 2A). In addition, IgG in 25 available patients' sera bound to DACH1 by WB using either HEK 293 DACH1-overexpressing lysate (FIG. 2B) or recombinant DACH1 polypeptide 282-758 (FIG. 2C). By indirect immunofluorescence on HEK293 DACH1-GFP transiently transfected cells, IgG in all available ANNA3-positive specimens, but in no control patients' specimens, were positive (FIG. 2D). Finally, preabsorption of serum with HEK293 DACH1-overexpressing lysate eliminated tissue binding of ANNA3-IgG, but did not affect the tissue binding of ANNA1-IgG (FIG. 2E).

### Clinical Characteristics

Non-uniform clinical information was available for 30 patients (Table 2): median age, 63.5 years (range, 49-88); 10 were male (33%). Of 22 patients with available information, 10 developed their neurological syndrome before cancer detection. The clinical phenotypes varied and included neuropathy in 12 (including small fiber neuropathy, sensorimotor neuropathies, polyradiculopathies and optic nerve involvement), cognitive difficulties or encephalitis in 11 patients (1 with seizures, 1 with auditory hallucinations alone), ataxia in 9, and autonomic involvement in 7. An additional two patients had diarrhea or esophageal spasms. Chorioretinopathy, optic neuropathy or Adie's pupils were documented in 3 individual patients. One of 2 patients with sensory neuronopathy had co-existing ANNA1-IgG. Two patients were assigned a discharge diagnosis of neurodegenerative disease based on clinical and radiological criteria or autopsy (#6 and #20) but a malignant neoplasm was found in both. Patients who had limited follow-up evaluation, or for whom incomplete information was provided by outside physicians, are described in Table 2. Six had interpretable CSF results (one excluded due to traumatic lumbar puncture [#2]); in 4 the CSF was inflammatory with either pleocytosis (3) or CSF-unique oligoclonal bands (1).

TABLE 2
ANNA3-IgG Seropositive patients' description.

| | | | | | | | CSF |
|---|---|---|---|---|---|---|---|
| | | | | | Serum/ | Co- | cell #/ |
| | | | | | CSF | existing | μL, |
| | Age/ | Neurologic | | | titer | neural | protein |
| | Sex | phenotype | Smoker | Cancer Type | (IFA) | Abs | mg/dL * |
| 1 | 55/M | Mild dysautonomia, | Yes | SCLC, lung | 1:30720/ | None | NA |
| | | ataxia (resolved | | squamous cell | NA | | |
| | | without treatment) | | | | | |
| 2 | 66/F | Stroke-like episodes | No | Breast, bladder | 1:15360/ | None | WBC 41, |
| | | | | (small cell) | 1:256 | | RBC |
| | | | | | | | 40000; |
| | | | | | | | protein |
| | | | | | | | 212 |
| 3 | 60/F | Small fiber | Yes | Ovarian | 1:240/NA | None | WBC 1; |
| | | neuropathy | | teratoma | | | protein |
| | | | | | | | 40, No |
| | | | | | | | OCB |
| 4 | 79/F | Ptosis, dysphagia | Yes | NA | 1:15360/ | None | NA |
| | | | | | 1:16 | | |
| 5 | 64/F | Weakness | Yes | Lung (NOS) | 1:30720/ | Serum:SOX1, | NA |
| | | | | | NA | 1:7680; | |
| | | | | | | VGCC P/Q, | |
| | | | | | | 0.49 nmol/L | |
| 6 | 65/M | Insidious cognitive | Yes | SCLC | 1:61440/ | None | NA |
| | | decline, | | | NA | | |
| | | neurodegenerative | | | | | |
| | | dementia suspected, | | | | | |
| | | ataxia | | | | | |
| 7 | 68/F | Subacute cognitive | No | Ovarian | 1:491520/ | None | NA |
| | | decline, dyskinesia, | | carcinoma | NA | | |
| | | neuropathy | | | | | |
| 8 | 82/M | Subacute cognitive | No | Poorly | 1:61440/ | None | NA |
| | | decline, ataxia | | differentiated | 1:16 | | |
| | | | | metastasis | | | |
| | | | | (unknown | | | |
| | | | | primary) | | | |
| 9 | 61/M | Ataxia, diplopia | No | Merkel cell | 1:480/1:4 | CSF: NIF | NA |
| | | | | | | (titer | |
| | | | | | | NA):heavy, | |
| | | | | | | alpha- | |
| | | | | | | internexin | |
| | | | | | | and light | |
| | | | | | | chain | |
| | | | | | | positive | |
| 10 | 63/F | Sensory | Yes | SCLC | 1:1920/ | Serum:VGCC | NA |
| | | neuronopathy, | | | NA | P/Q | |
| | | motor neuropathy, | | | | 0.13 nmol/L | |
| | | dysautonomia | | | | | |
| 11 | 76/F | Weakness, | NA | SCLC | 1:7680/ | None | NA |
| | | numbness, ataxia, | | | NA | | |
| | | dysautonomia, | | | | | |
| | | Lambert-Eaton | | | | | |
| | | myasthenic | | | | | |
| | | syndrome, SIADH | | | | | |
| 12 | 66/F | Sensorimotor | NA | Lung mass | 1:7680/ | Serum:GAD65 | NA |
| | | neuropathy, | | imaged | NA | 53.9 | |
| | | auditory | | | | nmol/L | |
| | | hallucinations, | | | | | |
| | | SIADH | | | | | |
| 13 | 49/F | Cognitive decline, | NA | Colon | 1:15360/ | Serum:GAD65 | NA |
| | | weakness | | | 1:128 | 0.28 | |
| | | | | | | nmol/L | |
| 14 | 51/M | Weakness from | No | None | 1:960/NA | Serum:ANNA1 | NA |
| | | motor neuropathy | | | | 1:7680, | |
| | | | | | | CRMP-5 | |
| | | | | | | 1:960, | |
| | | | | | | VGCC P/Q | |
| | | | | | | 0.05 nmol/L; | |
| | | | | | | Str 1:480 | |
| 15 | 60/F | Limbic encephalitis, | Yes | Lung mass | 1:7680/ | Serum:CRMP5 | WBC |
| | | seizures, optic | | imaged | NA | 1:15360, | 22; |
| | | neuropathy | | | | PCA2/MAP1B | Protein |
| | | | | | | 1:15360, | 181 |
| | | | | | | SOX1 | |
| 16 | 53/F | Cerebellar ataxia, | Yes | Hilar | 1:480/NA | Serum:gAChR | NA |
| | | subacute brainstem | | adenopathy | | 0.03 | |
| | | encephalopathy, | | imaged | | nmol/L | |
| | | chorioretinopathy | | | | | |
| 17 | 51/F | Eye movement | Yes | Lung | 1:960/NA | None | NA |
| | | disorder, dizziness, | | squamous & | | | |
| | | diarrhea | | adenocar- | | | |
| | | | | cinoma | | | |
| 18 | 62/F | Sensory ataxia from | Yes | SCLC, | 1:15360/ | Serum:ANNA1 | NA |
| | | sensory | | squamous | NA | 1:61440, | |
| | | neuronopathy, | | (mouth) | | CRMP-5 | |
| | | dysphagia, Adie's | | | | 1:15360 | |
| | | pupil, hoarseness | | | | | |
| 19 | 67/M | Esophageal spasm, | Yes | Gastric | 1:1920/ | None | NA |
| | | hoarseness | | neuroen- | NA | | |
| | | | | docrine | | | |
| 20 | 80/F | Parkinsonism, | No | Colon | 1:30720/ | None | NA |
| | | dementia, | | | NA | | |
| | | dysautonomia: | | | | | |
| | | Lewy body disease | | | | | |
| | | (autopsy confirmed) | | | | | |
| 21 | 88/F | Dementia, | NA | Breast | 1:1920/ | Serum:GAD65 | NA |
| | | neuropathy | | | NA | 4.6 | |
| | | | | | | nmol/L | |
| 22 | 63/F | Cerebellar ataxia, | Yes | Lung mass | 1:960/ | Serum:CRMP-5 | WBC |
| | | myelopathy, | | imaged | Neg | 1:7680 | normal, |
| | | sensorimotor and | | (biopsy non- | | CSF:CRMP5 | elevated |
| | | autonomic | | diagnostic) | | 1:1024 | protein, |
| | | neuropathy | | | | | Pos OCB |
| 23 | 70/F | Brainstem | NA | SCLC | 1:960/ | None | NA |
| | | syndrome, | | | 1:4 | | |
| | | neuropathy | | | | | |
| 24 | 60/M | Polyradiculoneu- | Yes | Metastatic | 1:960/ | None | WBC 17, |
| | | ropathy with | | neuroen- | 1:16 | | protein |
| | | quadriparesis, | | docrine | | | 110, No |
| | | hyponatremia | | (unknown | | | OCB |
| | | (SIADH) | | primary) | | | |
| 25 | 64/M | Subacute ataxia, | Yes | SCLC | 1:3840/64 | Serum:NIF | WBC |
| | | vertigo | | | | (heavy | 29; |
| | | | | | | chain) | protein |
| | | | | | | CSF:NIF | 97 |
| | | | | | | 1:64:heavy, | |
| | | | | | | light chain | |
| | | | | | | and alpha | |
| | | | | | | internexin | |
| 26 | 60/F | Subacute diplopia, | Yes | SCLC | 1:15360 | None | NA |
| | | ptosis, dysphagia, | | | | | |
| | | facial weakness, | | | | | |
| | | quadriparesis, | | | | | |
| | | sensory loss; onset | | | | | |
| | | after ICI | | | | | |
| 27 | 71/M | Weakness, urinary | Yes | Hyper- | 1:15360 | NA | NA |
| | | retention | | metabolic | | | |
| | | | | mass on FDG- | | | |
| | | | | PET (no | | | |
| | | | | available | | | |
| | | | | pathology) | | | |
| 28 | 59/F | Rapidly progressive | NA | metastatic | 1:1920/ | Serum:GAD65 | WBC 2; |
| | | weakness and | | neuro- | 1:16 | 0.45 | protein |
| | | encephalopathy | | endocrine | | nmol/L | 49 |
| | | | | (pleura, | | | |
| | | | | adrenal, | | | |
| | | | | pancreas) | | | |
| 29 | 80/M | Autoimmune | NA | None found | NA/1:8 | CSF:NIF, | NA |
| | | encephalitis and | | | | 1:128:heavy, | |
| | | polyneuropathy | | | | light chain | |
| | | | | | | and alpha | |
| | | | | | | internexin | |
| 30 | 63/F | Dermatomyositis | NA | SCLC | 1:960/NA | None | NA |

| | | | | Last |
|---|---|---|---|---|
| | Neuro-imaging | Immuno- | | follow-up |
| | & Other | therapy/Cancer | | (months)/ |
| | investigations | treatment | Improved | death |
| 1 | MRI Head: | No/Surgery, | NA | 125/Yes |
| | Normal | chemo-therapy | | |
| | | and radiation | | |
| 2 | MRI Head: Mild | No/Surgery | NA | 19/No |
| | atrophy; EEG: | | | |
| | normal | | | |
| 3 | MRI head & | IVIg, steroids, | Yes | 122/No |
| | spine: normal; | azathioprine/ | | |
| | Abnormal sweat | Surgery | | |
| | test and | | | |
| | autonomic reflex | | | |
| | screen | | | |
| 4 | NA | NA | NA | NA |
| 5 | NA | NA | NA | NA |
| 6 | FDG-PET - | NA | NA | 120/No |
| | suggests | | | |
| | Alzheimer | | | |
| | disease | | | |
| 7 | MRI head: | NA/Surgery, | NA | 12/No |
| | normal | chemotherapy | | |
| 8 | NA | NA | NA | 8/NA |
| 9 | NA | NA | NA | NA |
| 10 | NA | Steroids/NA | No | 2/NA |
| 11 | NA | NA/ | No | 13/Yes |
| | | Chemotherapy | | |
| 12 | MRI L spine: | IVIg/NA | Yes | NA |
| | normal; | | | |
| | NCS/EMG: | | | |
| | demyelinating | | | |
| | sensorimotor | | | |
| | neuropathy | | | |
| 13 | NA | NA | NA | NA |
| 14 | NA | IVIg, | Yes | NA |
| | | rituximab/NA | | |
| 15 | MRI head: Left | NA | NA | NA |
| | temporal T2- | | | |
| | hyperintensity | | | |
| | Brain biopsy left | | | |
| | temporal lobe: | | | |
| | inflammation; | | | |
| | EEG: left | | | |
| | temporal spikes | | | |
| 16 | MRI head: | NA | NA | 7/Yes |
| | brainstem and | | | |
| | cerebellum | | | |
| | diffuse T2- | | | |
| | hyperintensity | | | |
| 17 | NA | NA/Surgery | Yes | 105/Yes |
| 18 | MRI head and | NA | NA | NA |
| | spine: essentially | | | |
| | normal; | | | |
| | NCS/EMG: | | | |
| | sensory | | | |
| | neuronopathy | | | |
| 19 | NA | NA | NA | 6/NA |
| 20 | MRI head: | NA/Surgery | NA | 168/Yes |
| | normal; | | | |
| | Autopsy: | | | |
| | dementia with | | | |
| | Lewy bodies | | | |
| 21 | MRI head: | NA | NA | NA |
| | Normal; EEG: | | | |
| | diffuse slowing | | | |
| 22 | MRI head and | Steroids, | Yes | 10/Yes |
| | spine: essentially | cyclophos- | | |
| | normal; | phamide/NA | | |
| | NCS/EMG: | | | |
| | peripheral | | | |
| | neuropathy; | | | |
| | autonomic reflex | | | |
| | screen: | | | |
| | adrenergic and | | | |
| | cardiovagal | | | |
| | impairment | | | |
| 23 | NA | NA | NA | NA |
| 24 | MRI Lumbo- | Steroids/ | No | 18/No |
| | sacral-plexus: | chemotherapy | | |
| | enhancement; | then palliative | | |
| | MRI Head, C, T, | | | |
| | L spine: | | | |
| | negative. | | | |
| | NCS/EMG: | | | |
| | sensorimotor | | | |
| | polyradiculo- | | | |
| | neuropathy | | | |
| 25 | MRI head: | Steroids/NA | Yes | 7/No |
| | normal | | | |
| 26 | MRI head: | Steroids, IVIg/ | Yes; | 1/Yes |
| | leukoencephalopathy | Chemotherapy, | transitioned | |
| | secondary | ICI | to comfort | |
| | to radiation and | (atezolizumab), | care after | |
| | cerebellar brain | brain radiation | aspiration | |
| | metastasis; | for cerebellar | | |
| | NCS/EMG: | metastasis; | | |
| | sensorimotor | later, | | |
| | polyradiculo- | palliative | | |
| | neuropathy | | | |
| 27 | NA | NA | NA | NA |
| 28 | MRI brain: | PLEX/ | NA | NA |
| | normal; | chemotherapy | | |
| | NCS/EMG: | | | |
| | neuromuscular | | | |
| | junction defect | | | |
| | (unspecified) | | | |
| 29 | NA | NA | NA | NA |
| 30 | NA | None/ | Yes | 9/No |
| | | Chemotherapy & | | |
| | | ICI | | |
| | | (atezolizumab; | | |
| | | discontinued due | | |
| | | to worsening | | |
| | | dermatomyositis) | | |

Eight of 11 patients with treatment and outcome information showed improvement in their neurological syndrome; all 8 had either received immunotherapy or cancer treatment (chemotherapy, radiation or surgery). For two patients who had SCLC, neurological symptoms worsened or appeared for the first time during immune checkpoint inhibitor cancer immunotherapy (#26 and #30). One of those presented with classical dermatomyositis concurrent with the cancer diagnosis and had received just a single dose of atezolizumab (it was then discontinued). The other, with a multifocal neurological presentation, initially improved with corticosteroid and IVIg therapy, but aspiration pneumonia ensued and transitioned to palliative care.

### Oncological Findings

Ninety percent of the patients had evidence of cancer (27/30). In 22 the tumor was confirmed histopathologically; the other five had an imaged mass lesion highly suspicious for malignancy. In four patients, more than one malignancy was documented (SCLC and squamous cell carcinoma, 2; small-cell bladder carcinoma and breast adenocarcinoma, 1; bronchial adenocarcinoma and squamous cell carcinoma, 1). Neuroendocrine tumors accounted for 64% of all histopathologically confirmed neoplasms (14/22): SCLC, 9; neuroendocrine of unknown primary site, 2; bladder small-cell carcinoma, 1; Merkel cell carcinoma, 1; gastric neuroendocrine, 1. Other malignancies included: carcinomas of colon (2), breast (2), lung (1, adenocarcinoma) and ovary (1), a poorly differentiated carcinoma of unknown primary site (1) and an ovarian teratoma (1). All five patients lacking pathological confirmation of cancer had a lung mass, with or without hilar adenopathy (4), or FDG-avid PET mass (1). Two patients without a confirmed or presumed cancer, had no body imaging evidence of cancer. The neural autoantibody profile for both of those patients (#9 and #14) predicted a neuroendocrine cancer. For one patient (a smoker) no details were available regarding a cancer search.

### Additional Serological Findings

In cases with paired serum and CSF specimens, ANNA3-IgG was detected in both, except for patient #22 whose CSF was negative by IFA (possibly obscured by co-existing CRMP5-IgG) but was positive by DACH1-IgG CBA. All sera tested by DACH1 CBA were positive at 1:500 dilution or greater. Prozone effect was noted in one specimen (i.e., it was negative at 1:50 dilution). Fourteen patients had co-existing neural autoantibodies, most commonly predicting a neuroendocrine tumor: neuronal intermediate filament (NIF), 3; CRMP5, 3; voltage-gated calcium channel (VGCC) P/Q type, 3; ANNA1, 2; SOX1, 2; PCA2/MAP1B, 1. One patient had low-titer ganglionic acetylcholine receptor antibody and four patients had co-existing GAD65-IgG (exceeding 20 nmol/L in one patient) (Table 2).

### Immunoreactivity of Patients' Tumor Tissues

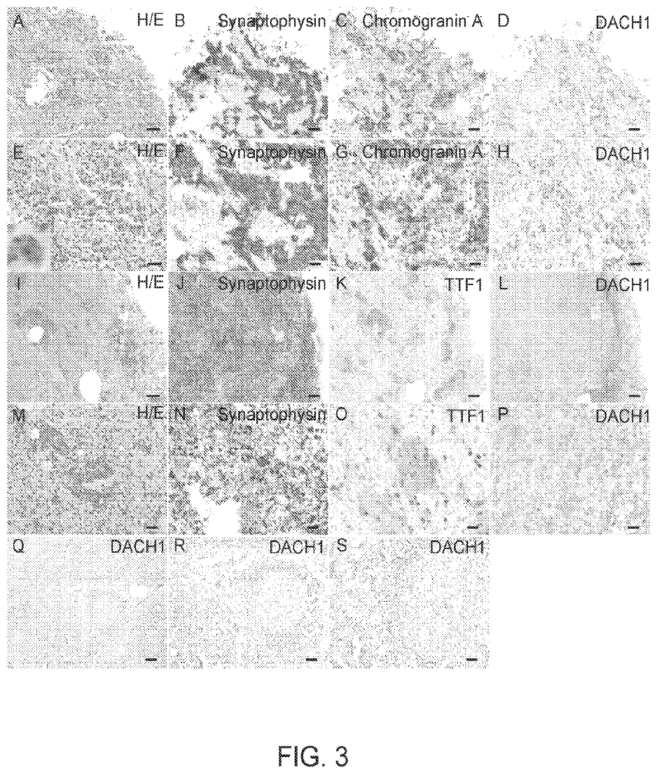

Lymph-node SCLC metastatic tumors were available from two ANNA3-IgG positive patients (#1 and #18) for immunohistochemical evaluation. In both, the neoplastic cells were compatible with a neuroendocrine tumor and expressed DACH1; DACH1 was not expressed by normal lymph-node tissue (FIG. 3).

Together these results demonstrate that a DACH1 polypeptide is the antigenic target of ANNA3-IgG, and that a DACH1 polypeptide fragment (e.g., a recombinant DACH1 polypeptide fragment) can be used to detect the presence of ANNA3-IgG in samples (e.g., serum samples).

### Example 2: Identifying ANNA3-Positive Neurological Autoimmune Disorders

A blood sample (e.g., serum) is obtained from a human having paraneoplastic neurological autoimmune disorder. The obtained sample is contacted with a fragment of a DACH1 polypeptide to form an ANNA3 autoantibody-fragment complex if the sample contains an ANNA3 autoantibody, and then examined for the presence of an ANNA3 autoantibody-fragment complex.

If ANNA3 autoantibody-fragment complexes are detected in the sample, then the human classified as having an ANNA3-positive paraneoplastic neurological autoimmune disorder.

### Example 3: Identifying ANNA3-Positive Neurological Autoimmune Disorders

A CSF sample is obtained from a human having a paraneoplastic neurological autoimmune disorder. The obtained sample is contacted with a fragment of a DACH1 polypeptide to form an ANNA3 autoantibody-fragment complex if the sample contains an ANNA3 autoantibody, and then examined for the presence of an ANNA3 autoantibody-fragment complex.

If ANNA3 autoantibody-fragment complexes are detected in the sample, then the human classified as having an ANNA3-positive paraneoplastic neurological autoimmune disorder.

### Example 4: Treating Neurological Autoimmune Disorders

A sample (e.g., serum) is obtained from a human having a paraneoplastic neurological autoimmune disorder. The sample is assessed for the presence or absence of ANNA3 antibodies by contacting the sample with a fragment of a DACH1 polypeptide to form an ANNA3 autoantibody-fragment complex if the sample contains an ANNA3 autoantibody, and then examined for the presence of an ANNA3 autoantibody-fragment complex.

When the presence of ANNA3 antibodies is detected in the sample, then the human is identified as having an ANNA3-positive paraneoplastic neurological autoimmune disorder, and is administered one or more ANNA3-positive paraneoplastic neurological autoimmune disorder treatments (e.g., cyclophosphamide, azathioprine, mycophenolate mofetil, methotrexate, and corticosteroids).

When the presence of ANNA3 antibodies is detected in the sample, then the human is identified as having an ANNA3-positive paraneoplastic neurological autoimmune disorder, and is not administered any of donepezil, memantine, riluzole, edaravone, haloperidol, and quetiapine.

### Example 5: Treating Neurological Autoimmune Disorders

A sample (e.g., serum) is obtained from a human having a paraneoplastic neurological autoimmune disorder. The sample is assessed for the presence or absence of ANNA3 antibodies by contacting the sample with a fragment of a DACH1 polypeptide to form an ANNA3 autoantibody-fragment complex if the sample contains an ANNA3 autoantibody, and then examined for the presence of an ANNA3 autoantibody-fragment complex.

When the absence of ANNA3 antibodies is detected in the sample, then the human is identified as having an ANNA3-negative paraneoplastic neurological autoimmune disorder, and is administered one or more ANNA3-negative paraneoplastic neurological autoimmune disorder treatments (e.g., cyclophosphamide, azathioprine, mycophenolate mofetil, methotrexate, and corticosteroids).

### Example 6: Identifying ANNA3-Positive Cancer

A blood sample (e.g., serum) is obtained from a human having cancer. The obtained sample is contacted with a fragment of a DACH1 polypeptide to form an ANNA3 autoantibody-fragment complex if the sample contains an ANNA3 autoantibody, and then examined for the presence of an ANNA3 autoantibody-fragment complex.

If ANNA3 autoantibody-fragment complexes are detected in the sample, then the human classified as having an ANNA3-positive cancer.

### Example 7: Identifying ANNA3-Positive Cancer

A CSF sample is obtained from a human having cancer. The obtained sample is contacted with a fragment of a DACH1 polypeptide to form an ANNA3 autoantibody-fragment complex if the sample contains an ANNA3 autoantibody, and then examined for the presence of an ANNA3 autoantibody-fragment complex.

If ANNA3 autoantibody-fragment complexes are detected in the sample, then the human classified as having an ANNA3-positive cancer.

### Example 8: Treating Cancer

A sample (e.g., serum) is obtained from a human having cancer. The sample is assessed for the presence or absence of ANNA3 antibodies by contacting the sample with a fragment of a DACH1 polypeptide to form an ANNA3 autoantibody-fragment complex if the sample contains an ANNA3 autoantibody, and then examined for the presence of an ANNA3 autoantibody-fragment complex.

When the presence of ANNA3 antibodies is detected in the sample, then the human is identified as having an ANNA3-positive cancer, and is subjected to radiation therapy and/or surgery, and/or is administered one or more anti-cancer agents.

When the presence of ANNA3 antibodies is detected in the sample, then the human is identified as having an ANNA3-positive cancer, and is not administered any immune checkpoint inhibitors (e.g., ipilimumab, nivolumab, pembrolizumab, atezolizumab, avelumab, durvalumab, and cemiplimab).

### Example 9: Treating Cancer

A sample (e.g., serum) is obtained from a human having cancer. The sample is assessed for the presence or absence of ANNA3 antibodies by contacting the sample with a fragment of a DACH1 polypeptide to form an ANNA3 autoantibody-fragment complex if the sample contains an ANNA3 autoantibody, and then examined for the presence of an ANNA3 autoantibody-fragment complex.

When the absence of ANNA3 antibodies is detected in the sample, then the human is identified as having an ANNA3-negative cancer, and is administered one or more immune checkpoint inhibitors (e.g., ipilimumab, nivolumab, pembrolizumab, atezolizumab, avelumab, durvalumab, and cemiplimab).

### Example 10: Exemplary Embodiments

Embodiment 1. A method for detecting an anti-neuronal nuclear antibody type 3 (ANNA3) autoantibody in a sample, wherein said method comprises:
(a) contacting said sample with a composition comprising a Dachshund homolog 1 (DACH1) polypeptide, wherein at least 5 percent of the polypeptide content of said composition is said DACH1 polypeptide to form an ANNA3 autoantibody-DACH1 polypeptide complex if said sample comprises said ANNA3 autoantibody; and
(b) detecting the presence or absence of said complex, wherein the presence of said complex indicates that said sample contains said ANNA3 autoantibody, and wherein the absence of said complex indicates that said sample lacks said ANNA3 autoantibody.

Embodiment 2. The method of embodiment 1, wherein said composition comprises a cell lysate obtained from a cell comprising exogenous nucleic acid encoding said DACH1 polypeptide and expressing said DACH1 polypeptide.

Embodiment 3. A method for detecting an ANNA3 autoantibody in a sample, wherein said method comprises:
(a) contacting said sample with a fragment of a DACH1 polypeptide to form an ANNA3 autoantibody-fragment complex if said sample comprises said ANNA3 autoantibody; and
(b) detecting the presence or absence of said complex, wherein the presence of said complex indicates that said sample contains said ANNA3 autoantibody, and wherein the absence of said complex indicates that said sample lacks said ANNA3 autoantibody.

Embodiment 4. The method of embodiment 3, wherein said DACH1 polypeptide fragment consists of the amino acid sequence set forth in any one of SEQ ID NOs:2-7.

Embodiment 5. The method of any one of embodiments 3-4, wherein said DACH1 polypeptide fragment is covalently linked to a detectable label.

Embodiment 6. The method of embodiment 5, wherein said detectable label is selected from the group consisting of tetramethylrhodamine isothiocyanate (TRITC), fluorescein isothiocyanate (FITC), a poly(His) tag, and a glutathione-S-transferase (GST) tag.

Embodiment 7. The method of any one of embodiments 1-6, wherein said complex is detecting using an immunological assay.

Embodiment 8. The method of any one of embodiments 1-7, wherein said sample is a serum sample.

Embodiment 9. The method of any one of embodiments 1-7, wherein said sample is a cerebrospinal fluid (CSF) sample.

Embodiment 10. The method of embodiment 8 or embodiment 9, wherein said sample is obtained from a human.

Embodiment 11. A method for assessing a mammal having an autoimmune neurological disorder, wherein said method comprises:
(a) determining the presence or absence of an ANNA3 autoantibody in a sample obtained from said mammal, wherein said determining comprises:
   (i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
   (ii) detecting the presence or absence of said complex;
(b) classifying said mammal as having an ANNA3-positive autoimmune neurological disorder if said complex is present; and
(c) classifying said mammal as having an ANNA3-negative autoimmune neurological disorder if said complex is absent.

Embodiment 12. The method of embodiment 11, wherein said composition comprises a cell lysate obtained from a cell comprising exogenous nucleic acid encoding said DACH1 polypeptide and expressing said DACH1 polypeptide.

Embodiment 13. A method for assessing a mammal having an autoimmune neurological disorder, wherein said method comprises:
(a) determining the presence or absence of an ANNA3 autoantibody in a sample obtained from said mammal, wherein said determining comprises:
   (i) contacting said sample with a fragment of a DACH1 polypeptide to form an ANNA3 autoantibody-fragment complex if said sample comprises said ANNA3 autoantibody, and
   (ii) detecting the presence or absence of said complex;
(b) classifying said mammal as having an ANNA3-positive autoimmune neurological disorder if said complex is present; and
(c) classifying said mammal as having an ANNA3-negative autoimmune neurological disorder if said complex is absent.

Embodiment 14. The method of embodiment 13, wherein said DACH1 polypeptide fragment consists of the amino acid sequence set forth in any one of SEQ ID NOs:2-7.

Embodiment 15. The method of any one of embodiments 11-14, wherein said mammal is a human.

Embodiment 16. The method of any one of embodiments 11-15, wherein said sample is a serum sample or a CSF sample.

Embodiment 17. The method of any one of embodiments 11-16, wherein said autoimmune neurological disorder is a paraneoplastic autoimmune neurological disorder.

Embodiment 18. The method of any one of embodiments 11-17, wherein said method comprises detecting the presence of said autoantibodies and classifying said mammal as having said ANNA3-positive autoimmune neurological disorder.

Embodiment 19. The method of any one of embodiments 11-17, wherein said method comprises detecting the absence of said autoantibodies and classifying said mammal as having said ANNA3-negative autoimmune neurological disorder.

Embodiment 20. A method for treating a mammal having an autoimmune neurological disorder, wherein said method comprises:
(a) determining the presence of an ANNA3 autoantibody in a sample obtained from said mammal, wherein said determining comprises:
   (i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
   (ii) detecting the presence of said complex, and
(b) administering an immunosuppressant to said mammal.

Embodiment 21. The method of embodiment 20, wherein said composition comprises a cell lysate obtained from a cell comprising exogenous nucleic acid encoding said DACH1 polypeptide and expressing said DACH1 polypeptide.

Embodiment 22. A method for treating a mammal having an autoimmune neurological disorder, wherein said method comprises:
(a) determining the presence of an ANNA3 autoantibody in a sample obtained from said mammal, wherein said determining comprises:
   (i) contacting said sample with a fragment of a DACH1 polypeptide to form an ANNA3 autoantibody-fragment complex if said sample comprises said ANNA3 autoantibody, and
   (ii) detecting the presence of said complex; and
(b) administering an immunosuppressant to said mammal.

Embodiment 23. The method of embodiment 21, wherein said DACH1 polypeptide fragment consists of the amino acid sequence set forth in any one of SEQ ID NOs:2-7.

Embodiment 24. The method of any one of embodiments 20-23, wherein said mammal is a human.

Embodiment 25. The method of any one of embodiments 20-24, wherein said sample is a serum sample or a CSF sample.

Embodiment 26. The method of any one of embodiments 20-25, wherein said autoimmune neurological disorder is a paraneoplastic autoimmune neurological disorder.

Embodiment 27. The method of any one of embodiments 20-26, wherein said immunosuppressant is selected from the group consisting of cyclophosphamide, rituximab, azathioprine, mycophenolate mofetil, methotrexate, and a corticosteroid.

Embodiment 28. A method for assessing a mammal having cancer, wherein said method comprises:
(a) determining the presence or absence of an ANNA3 autoantibody in a sample obtained from said mammal, wherein said determining comprises:
   (i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
   (ii) detecting the presence or absence of said complex;
(b) classifying said mammal as having an ANNA3-positive cancer if said complex is present; and
(c) classifying said mammal as having an ANNA3-negative cancer if said complex is absent.

Embodiment 29. The method of embodiment 28, wherein said composition comprises a cell lysate obtained from a cell comprising exogenous nucleic acid encoding said DACH1 polypeptide and expressing said DACH1 polypeptide.

Embodiment 30. A method for assessing a mammal having cancer, wherein said method comprises:
(a) determining the presence or absence of an ANNA3 autoantibody in a sample obtained from said mammal, wherein said determining comprises:
   (i) contacting said sample with a fragment of a DACH1 polypeptide to form an ANNA3 autoantibody-fragment complex if said sample comprises said ANNA3 autoantibody, and
   (ii) detecting the presence or absence of said complex;
(b) classifying said mammal as having an ANNA3-positive cancer if said complex is present; and
(c) classifying said mammal as having an ANNA3-negative cancer if said complex is absent.

Embodiment 31. The method of embodiment 30, wherein said DACH1 polypeptide fragment consists of the amino acid sequence set forth in any one of SEQ ID NOs:2-7.

Embodiment 32. The method of any one of embodiments 28-31, wherein said mammal is a human.

Embodiment 33. The method of any one of embodiments 28-32, wherein said sample is a serum sample or a CSF sample.

Embodiment 34. The method of any one of embodiments 28-33, wherein said cancer is selected from the group consisting of a lung cancer, a bladder cancer, a teratoma, an ovarian cancer, a Merkel cell cancer, a gastric cancer, a colon cancer, and a breast cancer.

Embodiment 35. The method of any one of embodiments 28-34, wherein said method comprises detecting the presence of said autoantibodies and classifying said mammal as having said ANNA3-positive cancer.

Embodiment 36. The method of any one of embodiments 28-34, wherein said method comprises detecting the absence of said autoantibodies and classifying said mammal as having said ANNA3-negative cancer.

Embodiment 37. A method for treating a mammal having cancer, wherein said method comprises:
(a) determining the presence of an ANNA3 autoantibody in a sample obtained from said mammal, wherein said determining comprises:
   (i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
   (ii) detecting the presence of said complex, and
(b) administering a cancer treatment to said mammal that is not an immune checkpoint inhibitor.

Embodiment 38. The method of embodiment 37, wherein said composition comprises a cell lysate obtained from a cell comprising exogenous nucleic acid encoding said DACH1 polypeptide and expressing said DACH1 polypeptide.

Embodiment 39. A method for treating a mammal having cancer, wherein said method comprises:
(a) determining the presence of an ANNA3 autoantibody in a sample obtained from said mammal, wherein said determining comprises:
   (i) contacting said sample with a fragment of a DACH1 polypeptide to form an ANNA3 autoantibody-fragment complex if said sample comprises said ANNA3 autoantibody, and
   (ii) detecting the presence of said complex; and
(b) administering a cancer treatment to said mammal that is not an immune checkpoint inhibitor.

Embodiment 40. The method of embodiment 39, wherein said DACH1 polypeptide fragment consists of the amino acid sequence set forth in any one of SEQ ID NOs:2-7.

Embodiment 41. The method of any one of embodiments 37-40, wherein said mammal is a human.

Embodiment 42. The method of any one of embodiments 37-41, wherein said sample is a serum sample or a CSF sample.

Embodiment 43. The method of any one of embodiments 37-42, wherein said cancer is selected from the group consisting of a lung cancer, a bladder cancer, a teratoma, an ovarian cancer, a Merkel cell cancer, a gastric cancer, a colon cancer, and a breast cancer.

Embodiment 44. The method of any one of embodiments 37-43, wherein said immune checkpoint inhibitor is selected from the group consisting of ipilimumab, nivolumab, pembrolizumab, atezolizumab, avelumab, durvalumab, and cemiplimab.

Embodiment 45. A method for treating a mammal having cancer, wherein said method comprises:
(a) determining the absence of an ANNA3 autoantibody in a sample obtained from said mammal, wherein said determining comprises:
   (i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
   (ii) detecting the absence of said complex, and
(b) administering an immune checkpoint inhibitor to said mammal.

Embodiment 46. The method of embodiment 45, wherein said composition comprises a cell lysate obtained from a cell comprising exogenous nucleic acid encoding said DACH1 polypeptide and expressing said DACH1 polypeptide.

Embodiment 47. A method for treating a mammal having cancer, wherein said method comprises:
(a) determining the absence of an ANNA3 autoantibody in a sample obtained from said mammal, wherein said determining comprises:
   (i) contacting said sample with a fragment of a DACH1 polypeptide to form an ANNA3 autoantibody-fragment complex if said sample comprises said ANNA3 autoantibody, and
   (ii) detecting the absence of said complex; and
(b) administering an immune checkpoint inhibitor to said mammal.

Embodiment 48. The method of embodiment 47, wherein said DACH1 polypeptide fragment consists of the amino acid sequence set forth in any one of SEQ ID NOs:2-7.

Embodiment 49. The method of any one of embodiments 45-48, wherein said mammal is a human.

Embodiment 50. The method of any one of embodiments 45-49, wherein said sample is a serum sample or a CSF sample.

Embodiment 51. The method of any one of embodiments 45-50, wherein said cancer is selected from the group consisting of a lung cancer, a bladder cancer, a teratoma, an ovarian cancer, a Merkel cell cancer, a gastric cancer, a colon cancer, and a breast cancer.

Embodiment 52. The method of any one of embodiments 45-51, wherein said immune checkpoint inhibitor is selected from the group consisting of ipilimumab, nivolumab, pembrolizumab, atezolizumab, avelumab, durvalumab, and cemiplimab.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting an anti-neuronal nuclear antibody type 3 (ANNA3) autoantibody in a sample, wherein said method comprises:
(a) contacting said sample with a composition comprising a Dachshund homolog 1 (DACH1) polypeptide, wherein at least 5 percent of the polypeptide content of said composition is said DACH1 polypeptide to form an ANNA3 autoantibody-DACH1 polypeptide complex if said sample comprises said ANNA3 autoantibody; and
(b) detecting the presence or absence of said complex, wherein the presence of said complex indicates that said sample contains said ANNA3 autoantibody, and wherein the absence of said complex indicates that said sample lacks said ANNA3 autoantibody.

2. The method of claim 1, wherein said composition comprises a cell lysate obtained from a cell comprising exogenous nucleic acid encoding said DACH1 polypeptide and expressing said DACH1 polypeptide.

3. A method for detecting an ANNA3 autoantibody in a sample, wherein said method comprises:
(a) contacting said sample with a fragment of a DACH1 polypeptide to form an ANNA3 autoantibody-fragment complex if said sample comprises said ANNA3 autoantibody; and
(b) detecting the presence or absence of said complex, wherein the presence of said complex indicates that said sample contains said ANNA3 autoantibody, and wherein the absence of said complex indicates that said sample lacks said ANNA3 autoantibody.

4. The method of claim 3, wherein said DACH1 polypeptide fragment consists of the amino acid sequence set forth in any one of SEQ ID NOs:2-7.

5. The method of claim 3, wherein said DACH1 polypeptide fragment is covalently linked to a detectable label.

6. The method of claim 5, wherein said detectable label is selected from the group consisting of tetramethylrhodamine isothiocyanate (TRITC), fluorescein isothiocyanate (FITC), a poly(His) tag, and a glutathione-S-transferase (GST) tag.

7. The method of claim 1, wherein said complex is detecting using an immunological assay.

8. The method of claim 1, wherein said sample is a serum sample.

9. The method of claim 1, wherein said sample is a cerebrospinal fluid (CSF) sample.

10. The method of claim 1, wherein said sample is obtained from a human.

11. A method for assessing a mammal having an autoimmune neurological disorder, wherein said method comprises:
(a) determining the presence or absence of an ANNA3 autoantibody in a sample obtained from said mammal, wherein said determining comprises:
(i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
(ii) detecting the presence or absence of said complex;
(b) classifying said mammal as having an ANNA3-positive autoimmune neurological disorder if said complex is present; and
(c) classifying said mammal as having an ANNA3-negative autoimmune neurological disorder if said complex is absent.

12. The method of claim 11, wherein said composition comprises a cell lysate obtained from a cell comprising exogenous nucleic acid encoding said DACH1 polypeptide and expressing said DACH1 polypeptide.

13-19. (canceled)

20. A method for treating a mammal having an autoimmune neurological disorder, wherein said method comprises:
(a) determining the presence of an ANNA3 autoantibody in a sample obtained from said mammal, wherein said determining comprises:
(i) contacting said sample with a composition comprising a DACH1 polypeptide, wherein at least 5 percent of the polypeptide content of said composition is said DACH1 polypeptide to form an ANNA3 autoantibody-DACH1 polypeptide complex if said sample comprises said ANNA3 autoantibody, and
(ii) detecting the presence of said complex, and
(b) administering an immunosuppressant to said mammal.

21. The method of claim 20, wherein said composition comprises a cell lysate obtained from a cell comprising exogenous nucleic acid encoding said DACH1 polypeptide and expressing said DACH1 polypeptide.

22. A method for treating a mammal having an autoimmune neurological disorder, wherein said method comprises:
(a) determining the presence of an ANNA3 autoantibody in a sample obtained from said mammal, wherein said determining comprises:
(i) contacting said sample with a fragment of a DACH1 polypeptide to form an ANNA3 autoantibody-fragment complex if said sample comprises said ANNA3 autoantibody, and
(ii) detecting the presence of said complex; and
(b) administering an immunosuppressant to said mammal.

23. The method of claim 21, wherein said DACH1 polypeptide fragment consists of the amino acid sequence set forth in any one of SEQ ID NOs:2-7.

24. The method of claim 20, wherein said mammal is a human.

25. The method of claim 20, wherein said sample is a serum sample or a CSF sample.

26. The method of claim 20, wherein said autoimmune neurological disorder is a paraneoplastic autoimmune neurological disorder.

27. The method of claim 20, wherein said immunosuppressant is selected from the group consisting of cyclophosphamide, rituximab, azathioprine, mycophenolate mofetil, methotrexate, and a corticosteroid.


SEQUENCE LISTING (7 sequences)

SEQ ID NO: 1 - protein, 758 aa, Homo sapiens
MAVPAALIPPTQLVPPQPPISTSASSSGTTTSTSSATSSPAPSIGPPASSGPTLFRPEPIASAAAAAATVTSTGGGGGGGGSGGGGGSSGNGGGGGGGGGGSNCNPNLAAASNGSGGGGGGISAGGGVASSTPINASTGSSSSSSSSSSSSSSSSSSSSSSSSCGPLPGKPVYSTPSPVENTPQNNECKMVDLRGAKVASFTVEGCELICLPQAFDLFLKHLVGGLHTVYTKLKRLEITPVVCNVEQVRILRGLGAIQPGVNRCKLISRKDFETLYNDCTNASSRPGRPPKRTQSVTSPENSHIMPHSVPGLMSPGIIPPTGLTAAAAAAAAATNAAIAEAMKVKKIKLEAMSNYHASNNQHGADSENGDMNSSVGSSDGSWDKETLPSSPSQGPQASITHPRMPGARSLPLSHPLNHLQQSHLLPNGLELPFMMMPHPLIPVSLPPASVTMAMSQMNHLSTIANMAAAAQVQSPPSRVETSVIKERVPDSPSPAPSLEEGRRPGSHPSSHRSSSVSSSPARTESSSDRIPVHQNGLSMNQMLMGLSPNVLPGPKEGDLAGHDMGHESKRMHIEKDETPLSTPTARDSLDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASTDSLRVLNDSLTPEIEADRSGGRTDAERTIQDGRLYLKTTVMY

SEQ ID NO: 2 - protein, 477 aa, synthetic construct
ASSRPGRPPKRTQSVTSPENSHIMPHSVPGLMSPGIIPPTGLTAAAAAAAAATNAAIAEAMKVKKIKLEAMSNYHASNNQHGADSENGDMNSSVGSSDGSWDKETLPSSPSQGPQASITHPRMPGARSLPLSHPLNHLQQSHLLPNGLELPFMMMPHPLIPVSLPPASVTMAMSQMNHLSTIANMAAAAQVQSPPSRVETSVIKERVPDSPSPAPSLEEGRRPGSHPSSHRSSSVSSSPARTESSSDRIPVHQNGLSMNQMLMGLSPNVLPGPKEGDLAGHDMGHESKRMHIEKDETPLSTPTARDSLDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASTDSLRVLNDSLTPEIEADRSGGRTDAERTIQDGRLYLKTTVMY

SEQ ID NO: 3 - protein, 706 aa, synthetic construct
MAVPAALIPPTQLVPPQPPISTSASSSGTTTSTSSATSSPAPSIGPPASSGPTLFRPEPIASAAAAAATVTSTGGGGGGGGSGGGGGSSGNGGGGGGGGGGSNCNPNLAAASNGSGGGGGGISAGGGVASSTPINASTGSSSSSSSSSSSSSSSSSSSSSSSSCGPLPGKPVYSTPSPVENTPQNNECKMVDLRGAKVASFTVEGCELICLPQAFDLFLKHLVGGLHTVYTKLKRLEITPVVCNVEQVRILRGLGAIQPGVNRCKLISRKDFETLYNDCTNASSRPGRPPKRTQSVTSPENSHIMPHSVPGLMSPGIIPPTGLTAAAAAAAAATNAAIAEAMKVKKIKLEAMSNYHASNNQHGADSENGDMNSSVGLELPFMMMPHPLIPVSLPPASVTMAMSQMNHLSTIANMAAAAQVQSPPSRVETSVIKERVPDSPSPAPSLEEGRRPGSHPSSHRSSSVSSSPARTESSSDRIPVHQNGLSMNQMLMGLSPNVLPGPKEGDLAGHDMGHESKRMHIEKDETPLSTPTARDSLDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASTDSLRVLNDSLTPEIEADRSGGRTDAERTIQDGRLYLKTTVMY

SEQ ID NO: 4 - protein, 558 aa, synthetic construct
MAVPAALIPPTQLVPPQPPISTSASSSGTTTSTSSATSSPAPSIGPPASSGPTLFRPEPIASAAAAAATVTSTGGGGGGGGSGGGGGSSGNGGGGGGGGGGSNCNPNLAAASNGSGGGGGGISAGGGVASSTPINASTGSSSSSSSSSSSSSSSSSSSSSSSSCGPLPGKPVYSTPSPVENTPQNNECKMVDLRGAKVASFTVEGCELICLPQAFDLFLKHLVGGLHTVYTKLKRLEITPVVCNVEQVRILRGLGAIQPGVNRCKLISRKDFETLYNDCTNASSRPGRPPKRTQSVTSPENSHIMPHSVPGLMSPGIIPPTGLTAAAAAAAAATNAAIAEAMKVKKIKLEAMSNYHASNNQHGADSENGDMNSSVDETPLSTPTARDSLDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASTDSLRVLNDSLTPEIEADRSGGRTDAERTIQDGRLYLKTTVMY

SEQ ID NO: 5 - protein, 504 aa, synthetic construct
MAVPAALIPPTQLVPPQPPISTSASSSGTTTSTSSATSSPAPSIGPPASSGPTLFRPEPIASAAAAAATVTSTGGGGGGGGSGGGGGSSGNGGGGGGGGGGSNCNPNLAAASNGSGGGGGGISAGGGVASSTPINASTGSSSSSSSSSSSSSSSSSSSSSSSSCGPLPGKPVYSTPSPVENTPQNNECKMVDLRGAKVASFTVEGCELICLPQAFDLFLKHLVGGLHTVYTKLKRLEITPVVCNVEQVRILRGLGAIQPGVNRCKLISRKDFETLYNDCTNASSRPGRPPKRTQSVTSPENSHIMPHSVPGLMSPGIIPPTDETPLSTPTARDSLDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASTDSLRVLNDSLTPEIEADRSGGRTDAERTIQDGRLYLKTTVMY

SEQ ID NO: 6 - protein, 47 aa, synthetic construct
QAASTDSLRVLNDSLTPEIEADRSGGRTDAERTIQDGRLYLKTTVMY

SEQ ID NO: 7 - protein, 16 aa, synthetic construct
ASSRPGRPPKRTQSVT